(19) United States
(12) Patent Application Publication
(10) Pub. No.: US 2011/0105477 A1
(43) Pub. Date: May 5, 2011

(54) COMPOSITIONS AND METHODS RELATING TO HIV PROTEASE INHIBITION

(75) Inventors: Heather A. Carlson, Ann Arbor, MI (US); Kelly L. Damm, Ann Arbor, MI (US); Kristen L. Meagher, Thousand Oaks, CA (US)

(73) Assignee: The Regents of the University of Michigan, Ann Arbor, MI (US)

(21) Appl. No.: 12/668,980

(22) PCT Filed: Sep. 12, 2008
(86) PCT No.: PCT/US08/76258

§ 371 Date: Nov. 15, 2010

Related U.S. Application Data
(60) Provisional application No. 60/972,505, filed on Sep. 14, 2007.

(51) Int. Cl. A61K 31/551; C07D 409/12; A61K 31/4709; A61P 31/18; C12N 9/99; C12Q 1/37; A61K 31/47; C07D 243/12; C07D 401/12; A61K 31/498; C07D 221/06; A61K 31/473; C07D 263/58; A61K 31/423; C07D 417/10; A61K 31/428; C07D 487/04; A61K 31/519; A61K 31/53

(52) U.S. Cl. 514/221; 546/178; 514/314; 435/184; 435/23; 514/311; 540/518; 544/354; 514/249; 546/79; 514/290; 548/221; 514/375; 548/159; 514/367; 544/263; 514/259.31; 544/251; 514/267; 544/184; 514/243

(57) ABSTRACT

The present invention relates to HIV protease, and methods for inhibiting the function of HIV protease. In particular, present invention provides compounds that inhibit or block the biological activity of HIVp, thereby causing the replication of the HIV virus to be inhibited or to terminate. These compounds, as well as pharmaceutical compositions that contain these compounds and optionally other anti-viral agents as active ingredients, are suitable for treating patients or hosts infected with the HIV virus, which is known to cause AIDS. The compounds and formulations also find use in diagnostic and research settings.

### COMPOSITIONS AND METHODS RELATING TO HIV PROTEASE INHIBITION

The present application claims priority to U.S. Provisional Patent Application Ser. No. 60/972,505, filed Sep. 14, 2007, which is herein incorporated by reference in its entirety.

This application was made with government support under Grant No. R01 GM065372 awarded by the National Institute of Health. The government has certain rights in this invention.

### FIELD OF THE INVENTION

The present invention relates to HIV protease and methods for inhibiting the function of HIV protease. In particular, present invention provides compounds that inhibit or block the biological activity of HIVp, thereby causing the replication of the HIV virus to be inhibited or to terminate. These compounds, as well as pharmaceutical compositions that contain these compounds and optionally other anti-viral agents as active ingredients, are suitable for treating patients or hosts infected with the HIV virus, which is known to cause AIDS. The compounds and formulations also find use in diagnostic and research settings.

### BACKGROUND OF THE INVENTION

Acquired immune deficiency syndrome or acquired immunodeficiency syndrome (AIDS or Aids) is a collection of symptoms and infections resulting from the specific damage to the immune system caused by the human immunodeficiency virus (HIV) in humans (see, e.g., Marx, J. L. (1982) Science 217 (4560): 618-621; herein incorporated by reference in its entirety), and similar viruses in other species (SIV, FIV, etc.). The late stage of the condition leaves individuals prone to opportunistic infections and tumors. Although treatments for AIDS and HIV exist to slow the virus' progression, there is no known cure. HIV is transmitted through direct contact of a mucous membrane or the bloodstream with a bodily fluid containing HIV, such as blood, semen, vaginal fluid, preseminal fluid, and breast milk. This transmission can come in the form of anal, vaginal or oral sex, blood transfusion, contaminated hypodermic needles, exchange between mother and baby during pregnancy, childbirth, or breastfeeding, or other exposure to one of the above bodily fluids.

Most researchers believe that HIV originated in sub-Saharan Africa during the twentieth century (see, e.g., Gao, et al., (1999) Nature 397 (6718): 436-441; herein incorporated by reference in its entirety); it is now a pandemic, with an estimated 38.6 million people now living with the disease worldwide. As of January 2006, the Joint United Nations Programme on HIV/AIDS (UNAIDS) and the World Health Organization (WHO) estimate that AIDS has killed more than 25 million people since it was first recognized on Jun. 5, 1981, making it one of the most destructive epidemics in recorded history. In 2005 alone, AIDS claimed an estimated 2.4-3.3 million lives, of which more than 570,000 were children. A third of these deaths are occurring in sub-Saharan Africa, retarding economic growth and destroying human capital. Antiretroviral treatment reduces both the mortality and the morbidity of HIV infection, but routine access to antiretroviral medication is not available in all countries (see, e.g., Palella, et al., (1998) N. Engl. J. Med 338 (13): 853-860; herein incorporated by reference in its entirety) HIV/AIDS stigma is more severe than that associated with other life-threatening conditions and extends beyond the disease itself to providers and even volunteers involved with the care of people living with HIV.

AIDS is the most severe acceleration of infection with HIV. HIV is a retrovirus that primarily infects vital organs of the human immune system such as CD4⁺ T cells (a subset of T cells), macrophages and dendritic cells. It directly and indirectly destroys CD4⁺ T cells. CD4⁺ T cells are required for the proper functioning of the immune system. When HIV kills CD4⁺ T cells so that there are fewer than 200 CD4⁺ T cells per microliter (μL) of blood, cellular immunity is lost, leading to the condition known as AIDS. Acute HIV infection progresses over time to clinical latent HIV infection and then to early symptomatic HIV infection and later to AIDS, which is identified on the basis of the amount of CD4⁺ T cells in the blood and the presence of certain infections.

In the absence of antiretroviral therapy, the median time of progression from HIV infection to AIDS is nine to ten years, and the median survival time after developing AIDS is only 9.2 months (see, e.g., Morgan, et al., (2002) AIDS 16 (4): 597-632; herein incorporated by reference in its entirety). However, the rate of clinical disease progression varies widely between individuals, from two weeks up to 20 years. Many factors affect the rate of progression. These include factors that influence the body's ability to defend against HIV such as the infected person's general immune function (see, e.g., Clerici, et al., (1996) AIDS Res. Hum. Retroviruses. 12 (11): 1053-1061; Morgan, et al., (2002) BMJ 324 (7331): 193-196; each herein incorporated by reference in their entireties). Older people have weaker immune systems, and therefore have a greater risk of rapid disease progression than younger people. Poor access to health care and the existence of coexisting infections such as tuberculosis also may predispose people to faster disease progression (see, e.g., Morgan, et al., (2002) AIDS 16 (4): 597-632; Gendelman, et al., (1986) Proc. Natl. Acad. Sci. U.S.A. 83 (24): 9759-9763; Bentwich, et al., (1995) Immunol. Today 16 (4): 187-191; each herein incorporated by reference in their entireties). The infected person's genetic inheritance plays an important role and some people are resistant to certain strains of HIV. An example of this is people with the CCR5-□32 mutation are resistant to infection with certain strains of HIV (see, e.g., Tang, et al., (2003) AIDS 17 (Suppl 4): S51-S60; herein incorporated by reference in its entirety). HIV is genetically variable and exists as different strains, which cause different rates of clinical disease progression (see, e.g., Quiñones-Mateu, et al., (1998) Virus Research 57 (1): 11-20; Campbell, et al., (2004) J. Biol. Chem. 279 (46): 48197-48204; Kaleebu, et al., (2002) J. Infect. Dis. 185 (9): 1244-1250; each herein incorporated by reference in their entireties). The use of highly active antiretroviral therapy prolongs both the median time of progression to AIDS and the median survival time.

There is currently no vaccine or cure for HIV or AIDS. The only known methods of prevention are based on avoiding exposure to the virus or, failing that, an antiretroviral treatment directly after a highly significant exposure, called post-exposure prophylaxis (PEP). PEP has a very demanding four week schedule of dosage. It also has very unpleasant side effects including diarrhea, malaise, nausea and fatigue.

What is needed are improved methods for treating HIV and AIDS. In particular, improved methods for inhibiting HIV Protease are needed.

### SUMMARY

The present invention relates to HIV protease, and methods for inhibiting the function of HIV protease. In particular, present invention provides compounds that inhibit or block the biological activity of HIVp, thereby causing the replication of the HIV virus to be inhibited or to terminate. These compounds, as well as pharmaceutical compositions that contain these compounds and optionally other anti-viral agents as active ingredients, are suitable for treating patients or hosts infected with the HIV virus, which is known to cause AIDS. The compounds and formulations also find use in diagnostic and research settings.

In experiments conducted during the course of development of embodiments for the present invention, a novel targeting region of HIVp was identified (“the newly identified HIVp binding site”) defined by a lower portion (e.g., defined by 184, V32, P81, T80, P79, and G78), an upper portion (e.g., defined by V56, 154, 147, G48, G49, and I50), and distal contacts (e.g., defined by V82 and the backbone atoms of V77, L33, and K55). It was found that small molecules can bind within the new site and prevent the “flaps” of the HIVp from properly folding to close the binding site; this blocks the formation of the reactive form of HIVp and inhibits the activity of the HIVp (e.g., inhibiting HIVp enzyme activity, inhibiting maturation of HIV particles, terminating replication of the HIV virus).

Accordingly, the present invention further provides methods of treating a disorder related to HIVp function comprising: administering to a subject an effective amount of at least one of the exemplary compounds of the present invention (see, e.g., Section II-Exemplary Compounds). In some embodiments, the disorder related to HIVp includes, but is not limited to, AIDS, AIDS Related Complex, HIV Infection, and associated complications and symptoms.

The compounds find use in treating a number of diseases and conditions in humans and animals and that find use in research, compound screening, and diagnostic applications. In some embodiments, compounds able to bind the newly identified HIVp binding site comprise the at least six of the following seven characteristics: three aromatic groups, two hydrophobic groups, one hydrogen bond donor group, and one hydrogen bond acceptor group. In some embodiments, the compounds are described by one or more of the compounds provided in FIGS. 1 and 2, equivalents and/or functional equivalents thereof.

In certain embodiments, the compounds are described by the following formulas:

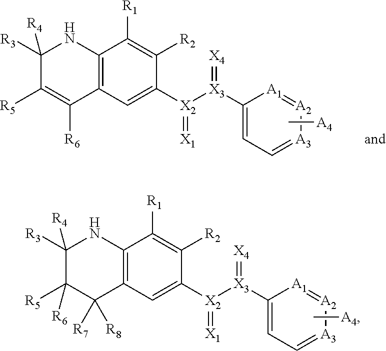

including salts, esters, and prodrugs thereof, and including both R and S enantiomeric forms and racemic mixtures thereof, wherein R1 and R2 are independently functionalized with hydrogen, hydrogen bond acceptors or donors (e.g., OH), R3, R4, R5, R6, R7 and R8 can be independently functionalized with small hydrophobic groups (e.g., methyl, ethyl, iodo, bromo, and chloro—R5, R6, R7 or R8 can also have a small hydrophilic group such as hydroxyl), where X1 and X4 are present or absent and, if present, are independently selected from O and S, and where X2 and X3 are independently selected from O, C, S, SO₂, PO₂, or NH, where A1, A2, and A3 are independently selected from C and N, and where A4 is present or absent, and if present, is

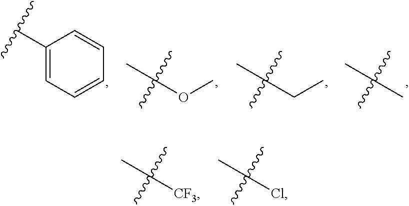

or a solubilizing group

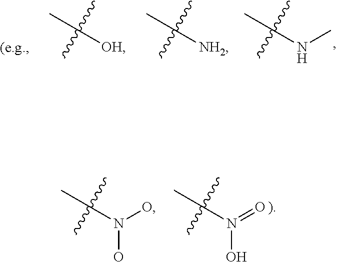

In some embodiments, the compound is described by any of the following formulas:

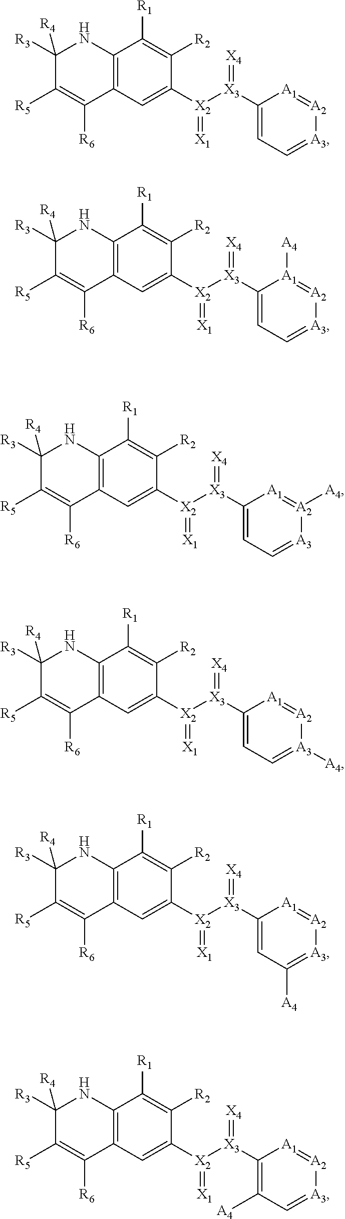

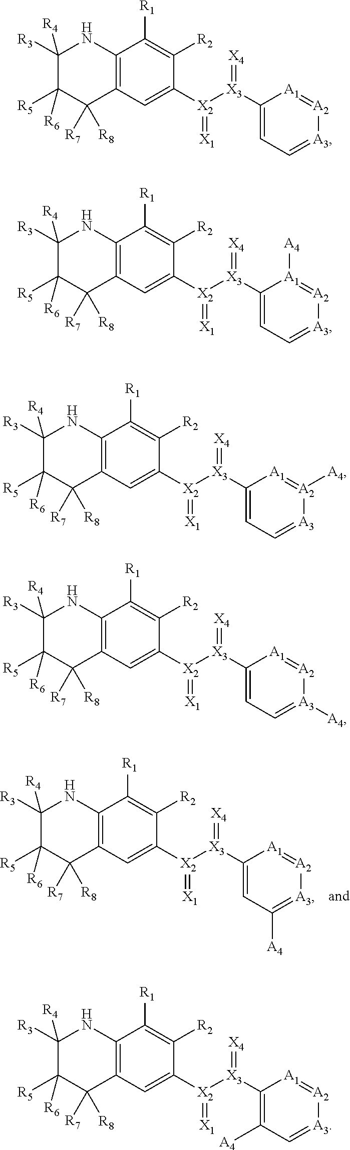

In some embodiments, the compounds comprise three aromatic groups, two hydrophobic groups, one hydrogen bond donor group, and one hydrogen bond acceptor group. In some embodiments, the position of groups is defined by ranges of distances and positions with respect to one another as described in Tables 1 and 2.

In some embodiments, the compounds are described by the formula C1C2-A-B, including salts, esters, and prodrugs thereof, and including both R and S enantiomeric forms and racemic mixtures thereof. In some embodiments, the compound comprises three aromatic groups, one at position C1, one at position C2, one at position B, two hydrophobic groups, one hydrogen bond donor group, and one hydrogen bond acceptor group, wherein A provides a covalent linker connecting C1C2 and B. In some embodiments, the C1 group is not aromatic. In some embodiments, the position of groups is defined by ranges of distances and positions with respect to one another as described in Tables 1 and 2. In some embodiments wherein the compound is described by the formula: C1C2-A-B, the compound has at least six of the seven characteristics of the newly identified HIVp binding site (e.g., an aromatic group at position C1, an aromatic group at position C2, an aromatic group at position B, two hydrophobic groups, one hydrogen bond donor group, and one hydrogen bond acceptor group). In some embodiments, the compound described by the formula C1C2-A-B has all seven of these characteristics. In some embodiments, the compound described by the formula C1C2-A-B has six of the seven characteristics. In some embodiment where the compound described by C1C2-A-B has only six of the seven characteristics, the C1 group is not aromatic.

In some embodiments, “C1C2” is described by the following formulas:

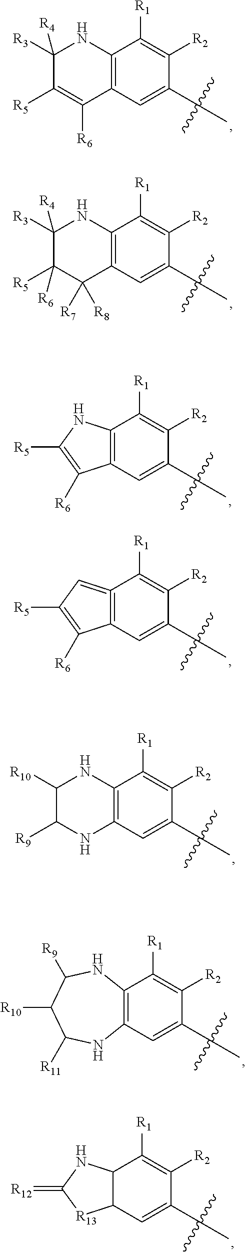

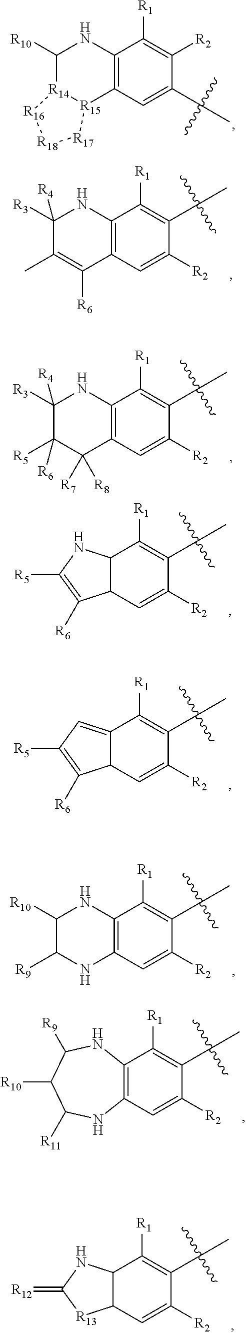

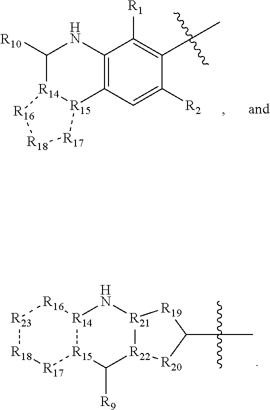

In some embodiments, R1 and R2 are independently hydrogen, hydrogen-bond acceptors or donors (e.g., OH). In some embodiments, R3, R4, R5, R6, R7 and R8 can be independently functionalized with small hydrophobic groups (e.g., methyl, ethyl, iodo, bromo, and chloro—R5, R6, R7 or R8 can also have a small hydrophilic group such as hydroxyl). In some embodiments, R3 and R4 are each methyl, and R5 and R6 are each hydrogen. In some embodiments, R9, R10 and R11 are independently halogen (e.g., Cl, I, Br, F), C-(halogen (e.g., Cl, I, Br, F))₃, CF₃, CI₃, CBr₃, CCl₃, COO⁻, COOH, ketone, hydrogen and methyl. In some embodiments, R12 and R13 is independently O or S. In some embodiments, R14 and 15 is either C═C or CH—CH. In some embodiments, R16, R17, R18, and R23 are independently present or absent, and if present are independently COO⁻, COOH, ketone, hydrogen, halogen (e.g., Cl, I, Br, F), C-(halogen (e.g., Cl, I, Br, F))₃, CF₃, CI₃, CBr₃, CCl₃, C, CH, CH2, and methyl. In some embodiments, R19 and R20 are independently C, CH, CH2, N, and NH. In some embodiments, R21 and R22 is C, CH and N.

In some embodiments, C1C2 is described by the following structures:

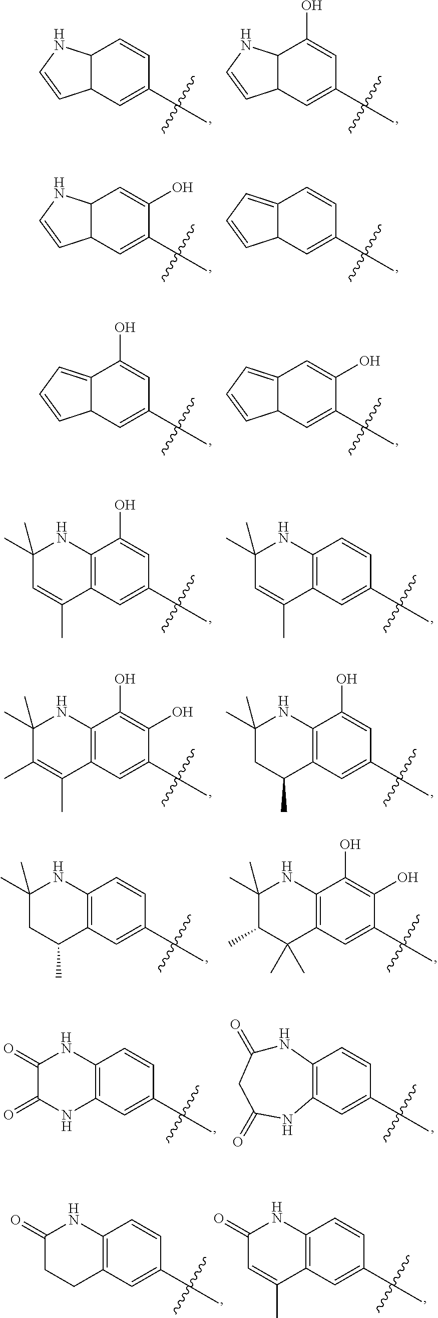

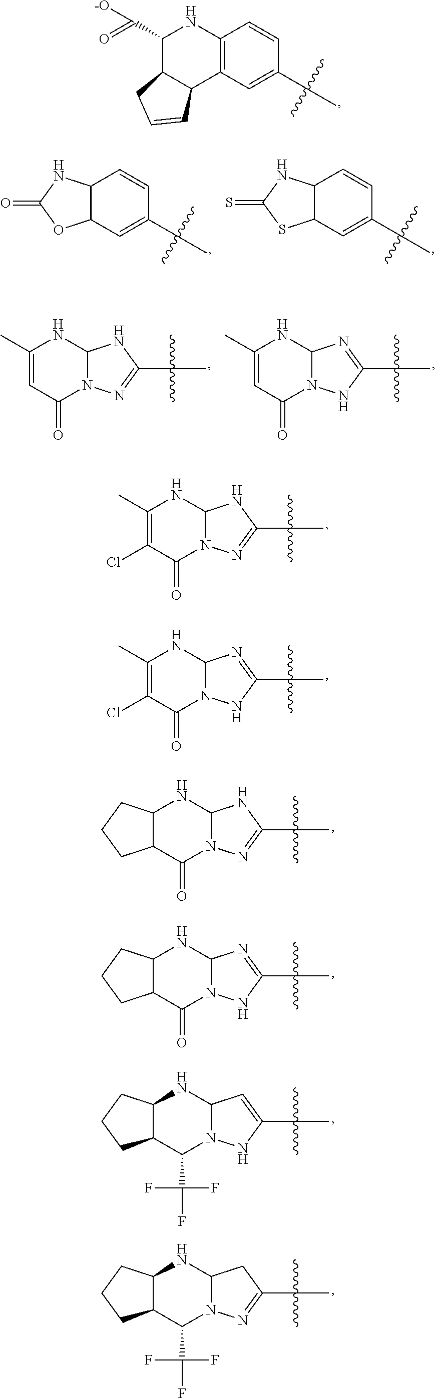

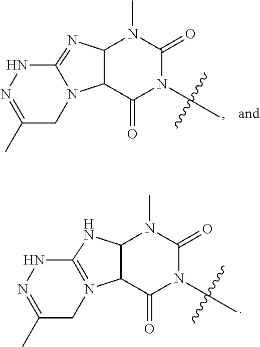

In some embodiments, “B” includes, but is not limited to, the following structures:

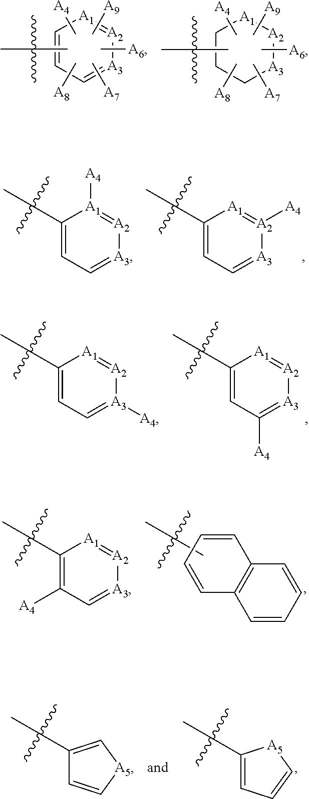

where A1, A2, and A3 are independently selected from C and N, where A4, A6, A7, A8, and A9 are independently present or absent, and if present, are independently halogen (e.g., Cl, I, Br, F), C-(halogen (e.g., Cl, I, Br, F))₃, CF₃, CI₃, CBr₃, CCl₃,

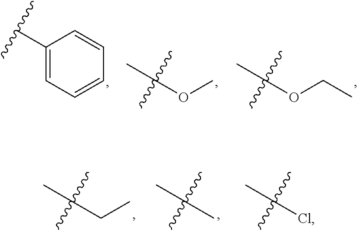

or a solubilizing group

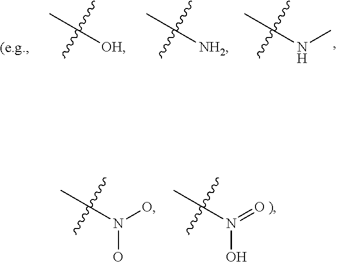

and where A5 is selected from O, C, S, or NH. In some embodiments, examples of “B” include, but are not limited to,

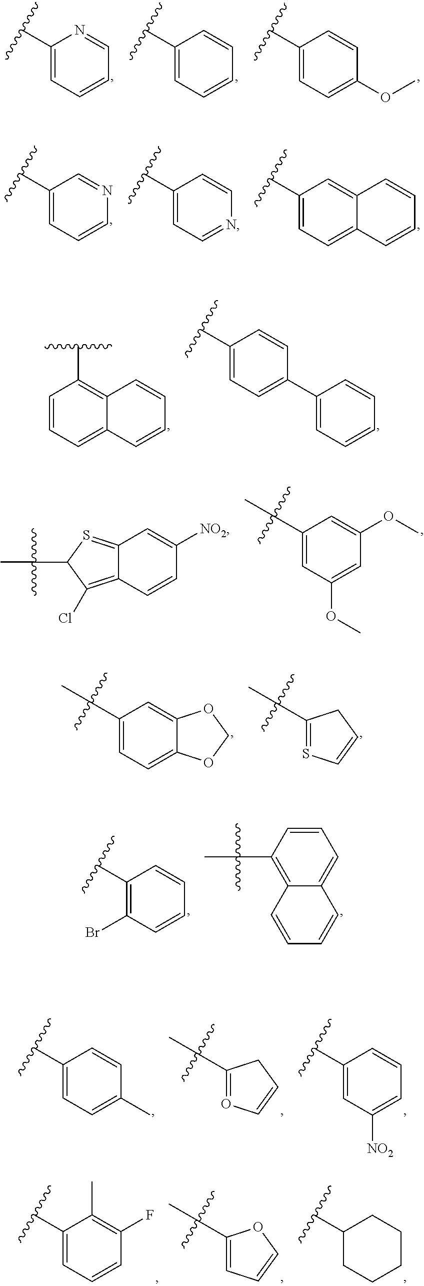

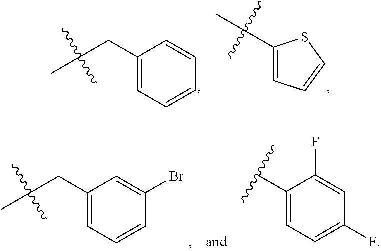

In some embodiments, “B” is a non-aromatic cyclical compound. Examples include, but are not limited to,

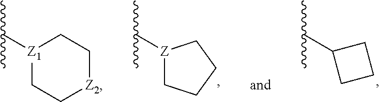

where Z, Z1 and Z2 are independently C or N. In some embodiments, Z1 and Z2 are the same. In some embodiments, Z1 and Z2 are different. In some embodiments, B is an aromatic ring. In some embodiments, the aromatic ring is benzene.

In some embodiments, the B subgroup is an aromatic ring substituted with one or more of the following chemical moieties: hydroxyl, methoxyl, ethoxyl, methyl, ethyl, isopropyl, amine, methylamine, and halides. In some embodiments, larger substitution groups are included at the para position of the aromatic group. In some embodiments, the solubility of the compound is improved by having a hydrophilic (e.g., OH) group positioned at the para position of the aromatic ring.

In some embodiments, “A” is a covalent linker connecting “C1C2” and “B.” In some embodiments, examples of “A” include, but are not limited to, chemical moieties comprising esters, ethers, amides, amines, ketones, thioesters, thioethers, thioamides, thioketones and short alkyl chains. In some embodiments, “A” includes, but is not limited to, the following structures:

In some embodiments, “A” is a covalent linker connecting “C1C2” and “B.” In some embodiments, examples of “A” include, but are not limited to, chemical moieties comprising esters, ethers, amides, amines, ketones, thioesters, thioethers, thioamides, thioketones and short alkyl chains. In some embodiments, “A” includes, but is not limited to, the following structures:

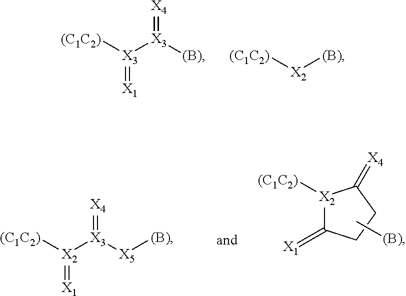

where X1 and X4 are present or absent and, if present, are independently selected from O and S, and where X2, X3 and X5 are independently selected from O, C, S, SO₂, PO₂, N, or NH.

In some embodiments, examples of “A” include, but are not limited to, chemical moieties comprising sulfonates, sulfonamides, phosphonates, and/or phosphamides. In some embodiments, use of one or more phosphate groups comprising ³¹P and/or ³²P within “A” permits improved labeling for detection purposes (e.g., NMR detection; scintillation detection). In some embodiments, other detectable atoms may be incorporated within the compound for detection and/or labeling purposes. In some embodiments, “A” is a phosphate linkage. In some embodiments, “A” is a phosphate linkage. In some embodiments, “A” is a sulfate linkage. In some embodiments, “A” includes, but is not limited to, the following structures:

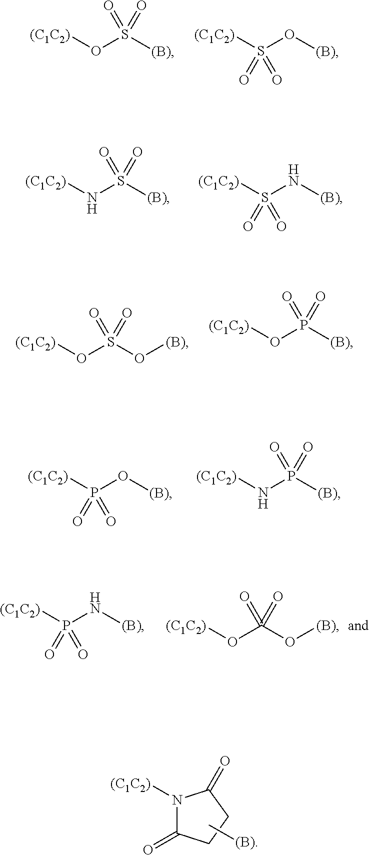

In some embodiments, C1C2-A-B is described by one of the following compounds, including salts, esters, and prodrugs thereof, and including both R and S enantiomeric forms and racemic mixtures thereof:

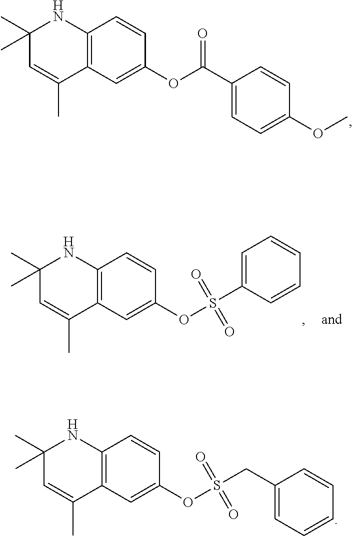

In some embodiments, C1C2-A-B is described by one of the compounds described in Table 4.

In some embodiments, the compounds are co-administered with agents known to treat HIV and/or HIV related disorders (e.g., antibiotics (e.g., Trimethoprim/sulfamethoxazole, pentamidine), glucocorticoids (e.g., prednisone), antifungals (e.g., amphotericin B, flucytosine, clotrimazole), antivirals (e.g., zidovudine, lamivudine, abacavir, nevirapine, ganciclovir, tenofovir), protease inhibitors (e.g., lopinavir, ritonavir, indinavir), antiparasitics (e.g., pyrimethamine). Other examples of agents known to treat HIV and/or HIV related disorders include, but are not limited to, nucleotide reverse transcriptase inhibitor (e.g., zidovudine (AZT); didanosine (ddI); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)—FTC]; beta-L-FD4 (also called beta-L-D4C and named beta-L-2′,3′-dicleoxy-5-fluoro-cytidene); DAPD, the purine nucleoside, (−)-beta-D-2,6,-diamino-purine dioxolane; and lodenosine (FddA)), a non-nucleoside reverse transcriptase inhibitor (e.g., nevirapine (BI-RG-587); delaviradine (BHAP, U-90152); efavirenz (DMP-266) a benzoxazin-2-one; PNU-142721, a furopyridine-thio-pyrimide; 5-(3,5-dichlorophenyl)-thio-4-isopropyl-1-(4-pyridyl)methyl-I H-imidazol-2-ylmethyl carbonate; MKC442 (1-(ethoxymethyl)-5-(1-methylethyl)-6-(phenylmethyl)-(2,4(1H,3H)-pyrimidi-nedione); and (+)-calanolide A (NSC-675451) and B, coumarin derivatives), a protease inhibitor (e.g., saquinavir (Ro 31-8959); ritonavir (ABT-538); nelfnavir (AG-1343); amprenavir (141W94); lasinavir (BMS-234475); DMP-450; ABT-378; and AG-1549), and an antiviral agent (e.g., hydroxyurea, ribavirin, IL-2, IL-12, pentafuside, and hydroxyurea (Droxia), IL-2, and pentafuside (DP-178, T-20), and integration inhibitors (e.g., “raltegravir, MK-0518,” “elvitegravir, GS 9137”, “364735,” U.S. Patent Application Publication Nos. 20060134612, 20060035245, and 20050261336; Bonnenfant, et al., 2004, J. Virol. 78(11): 5728-5736; Espeseth, et al., 2000, PNAS 97:11244-11249; Hazuda, et al., 2000 Science 287:646-650). In some embodiments, one or more of the compounds of the present invention are provided with one or more agents known to treat HIV and/or HIV related disorders in a “cocktail” for purposes of delivery to a subject.

In some embodiments, compounds of the present invention, and other potentially useful compounds, are screened for an ability to displace compounds bound in the newly identified HIVp binding site. In some embodiments, compounds of the present invention, and other potentially useful compounds, are screened for an ability to inhibit the function of HIVp (e g., inhibiting HIVp enzyme activity, inhibiting maturation of HIV particles, terminating replication of the HIV virus) through binding the newly identified HIVp binding site. In some embodiments, compounds of the present invention, and other potentially useful compounds, are screened for an ability to treat disorders associated with HIVp (e.g., AIDS, AIDS Related Complex, HIV Infection, and associated complications and symptoms) through binding the newly identified HIVp binding site.

In certain embodiments, the present invention provides compositions comprising one or more therapeutic agents known to treat disorders associated with HIV protease, and one or more compounds known to bind a region of HIV protease defined by the following residues: I84, V32, P81, T80, P79, G78, V56, 154, 147, G48, G49, and I50. In some embodiments, the compound is one of the compounds of the present invention described in Section II—Exemplary Compounds. The compositions are not limited to particular therapeutic agents.

Examples of therapeutic agents include, but are not limited to, nucleotide reverse transcriptase inhibitors, a non-nucleoside reverse transcriptase inhibitors, a protease inhibitors, and an antiviral agents. Further examples of therapeutic agents include, but are not limited to, zidovudine (AZT); didanosine (ddI); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)—FTC]; beta-L-FD4 (also called beta-L-D4C and named beta-L-2′,3′-dicleoxy-5-fluoro-cytidene); DAPD, the purine nucleoside, (−)-beta-D-2,6,-diamino-purine dioxolane; and lodenosine (FddA)), nevirapine (BI-RG-587); delaviradine (BHAP, U-90152); efavirenz (DMP-266) a benzoxazin-2-one; PNU-142721, a furopyridine-thio-pyrimide; 5-(3,5-dichlorophenyl)-thio-4-isopropyl-1-(4-pyridyl)methyl-I H-imidazol-2-ylmethyl carbonate; MKC442 (1-(ethoxymethyl)-5-(1-methylethyl)-6-(phenylmethyl)-(2,4(1H,3H)-pyrimidi-nedione); and (+)-calanolide A (NSC-675451) and B, coumarin derivatives), saquinavir (Ro 31-8959); ritonavir (ABT-538); nelihavir (AG-1343); amprenavir (141W94); lasinavir (BMS-234475); DMP-450; ABT-378; and AG-1549), hydroxyurea, ribavirin, IL-2, IL-12, pentafuside, and hydroxyurea (Droxia), IL-2, and pentafuside (DP-178, T-20).

In certain embodiments, the present invention provides methods for inhibiting the activity of HIV protease, comprising binding a compound within a region of HIV protease defined by the following residues: I84, V32, P81, T80, P79, G78, V56, I54, I47, G48, G49, and I50. In some embodiments, the compound is one of the compounds of the present invention described in Section II—Exemplary Compounds.

In certain embodiments, the present invention provides methods for treating disorders associated with HIV protease, comprising administering to a subject suffering from an HIV protease associated disorder a therapeutic amount of one or more compounds known to bind a region of HIV protease defined by the following residues: I84, V32, P81, T80, P79, G78, V56, I54, I47, G48, G49, and I50. In some embodiments, the compound is one of the compounds of the present invention described in Section II—Exemplary Compounds. In some embodiments, the disorder associated with HIV protease is selected from the group consisting of: AIDS, AIDS Related Complex, HIV Infection, complications associated with AIDS, complications associated with HIV Infection, complications associated with AIDS Related Complex.

In certain embodiments, the present invention provides methods of screening for compounds able to bind a region of HIV protease defined by one or more of the following residues: I84, V32, P81, T80, P79, G78, V56, I54, I47, G48, G49, and I50, comprising administering a candidate compound to a sample comprising HIV protease, and detecting binding of the compound within the HIV protease region.

### BRIEF DESCRIPTION OF THE DRAWINGS

FIGS. 1 and 2 show various compounds in embodiments of the invention.

FIG. 3 shows solvent-mapping probes docked into the new HIVp binding site on the right-side monomer of the HIVp dimer; they occupy the position taken by the flap of the opposite monomer and prevent closure of the left-side flap. Both monomers contain identical binding sites and flap closure can be prevented on one or both sides to inhibit HIVp.

FIG. 4 shows the new HIVp binding site and the individual residues which define it.

FIG. 5 shows pharmacophore models for the new HIVp binding site.

FIG. 6 shows a pharmacophore model for the new HIVp binding site.

FIG. 7 shows three of the NMR shifts involved G48, G52, and I54 which are central to the new binding site. The fourth shift of the side chain of Q58 indicates some structural rearrangements of HIVp or a secondary interaction that is possible at high concentrations.

### DEFINITIONS

To facilitate an understanding of the present invention, a number of terms and phrases are defined below.

As used herein, the term “therapeutically effective dose” is meant a dose that produces the desired effect for which it is administered. The exact dose will depend on the purpose of the treatment, and will be ascertainable by one skilled in the art using known techniques (see, e.g., Lloyd (1999) The Art, Science and Technology of Pharmaceutical Compounding).

As used herein, the terms “HIV protease,” “HIVp,” and “HIV aspartyl protease” are used interchangeably and refer to the aspartyl protease encoded by the human immunodeficiency virus type 1 or 2. In some embodiments of this invention, these terms refer to the human immunodeficiency virus type 1 aspartyl protease.

As used herein, the term “HIV infection” generally encompasses infection of a host, particularly a human host, by the human immunodeficiency virus (HIV) family of retroviruses including, but not limited to, HIV I, HIV II, HIV III (also known as HTLV-II, LAV-1, LAV-2), and the like. “HIV” can be used herein to refer to any strains, forms, subtypes, clades and variations in the HIV family. Thus, treating HIV infection will encompass the treatment of a person who is a carrier of any of the HIV family of retroviruses or a person who is diagnosed of active AIDS, as well as the treatment or prophylaxis of the AIDS-related conditions in such persons. A carrier of HIV may be identified by any methods known in the art. For example, a person can be identified as an HIV carrier on the basis that the person is anti-HIV antibody positive, or is HIV-positive, or has symptoms of AIDS. That is, “treating HIV infection” should be understood as treating a patient who is at any one of the several stages of HIV infection progression, which, for example, include acute primary infection syndrome (which can be asymptomatic or associated with an influenza-like illness with fevers, malaise, diarrhea and neurologic symptoms such as headache), asymptomatic infection (which is the long latent period with a gradual decline in the number of circulating CD4⁺ T cells), and AIDS (which is defined by more serious AIDS-defining illnesses and/or a decline in the circulating CD4 cell count to below a level that is compatible with effective immune function). In addition, “treating or preventing HIV infection” will also encompass treating suspected infection by HIV after suspected past exposure to HIV by e.g., contact with HIV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HIV infection” may also encompass treating a person who has not been diagnosed as having HIV infection but is believed to be at risk of infection by HIV.

As used herein, the term “symptoms associated with AIDS,” “complications associated with AIDS,” “symptoms associated with HIV,” “complications associated with HIV,” “symptoms associated with HIV protease,” “complications associated with HIV protease” or similar terms, refers to bacterial infections, Kaposi's sarcoma, cervical cancer, lymphoma, fever, weight loss, pulmonary illnesses (e.g., pneumocystis pneumonia, tuberculosis), gastro-intestinal illnesses (e.g., esophagitis), chronic wasting, and neurological illnesses (e.g., toxoplasmosis, progressive multifocal leukoencephalopahthy, AIDS dementia complex, cryptococcal meningitis).

The term “treating AIDS” means treating a patient who exhibits more serious AIDS-defining illnesses and/or a decline in the circulating CD4 cell count to below a level that is compatible with effective immune function. The term “treating AIDS” also encompasses treating AIDS-related conditions, which means disorders and diseases incidental to or associated with AIDS or HIV infection such as AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

As used herein, the term “preventing AIDS” means preventing in a patient who has HIV infection or is suspected to have HIV infection or is at risk of HIV infection from developing AIDS (which is characterized by more serious AIDS-defining illnesses and/or a decline in the circulating CD4 cell count to below a level that is compatible with effective immune function) and/or AIDS-related conditions.

The term “nucleoside and nucleotide reverse transcriptase inhibitors” (“NRTI”s) as used herein means nucleosides and nucleotides and analogues thereof that inhibit the activity of HIV-1 reverse transcriptase, the enzyme which catalyzes the conversion of viral genomic HIV-1 RNA into proviral HIV-1 DNA. Typical suitable NRTIs include zidovudine (AZT) available under the RETROVIR tradename from Glaxo-Wellcome Inc., Research Triangle, N.C. 27709; didanosine (ddI) available under the VIDEX tradename from Bristol-Myers Squibb Co., Princeton, N.J. 08543; zalcitabine (ddC) available under the HIVID tradename from Roche Pharmaceuticals, Nutley, N.J. 07110; stavudine (d4T) available under the ZERIT trademark from Bristol-Myers Squibb Co., Princeton, N.J. 08543; lamivudine (3TC) available under the EPIVIR tradename from Glaxo-Wellcome Research Triangle, N.C. 27709; abacavir (1592U89) disclosed in WO96/30025 and available under the ZIAGEN trademark from Glaxo-Wellcome Research Triangle, N.C. 27709; adefovir dipivoxil [bis(POM)-PMEA] available under the PREVON tradename from Gilead Sciences, Foster City, Calif 94404; lobucavir (BMS-180194), a nucleoside reverse transcriptase inhibitor disclosed in EP-0358154 and EP-0736533 and under development by Bristol-Myers Squibb, Princeton, N.J. 08543; BCH-10652, a reverse transcriptase inhibitor (in the form of a racemic mixture of BCH-10618 and BCH-10619) under development by Biochem Pharma, Laval, Quebec H7V, 4A7, Canada; emitricitabine [(−)—FTC] licensed from Emory University under Emory Univ. U.S. Pat. No. 5,814,639 and under development by Triangle Pharmaceuticals, Durham, N.C. 27707; beta-L-FD4 (also called beta-L-D4C and named beta-L-2′,3′-dicleoxy-5-fluoro-cytidene) licensed by Yale University to Vion Pharmaceuticals, New Haven Conn. 06511; DAPD, the purine nucleoside, (−)-beta-D-2,6,-diamino-purine dioxolane disclosed in EP 0656778 and licensed by Emory University and the University of Georgia to Triangle Pharmaceuticals, Durham, N.C. 27707; and lodenosine (FddA), 9-(2,3-dideoxy-2-fluoro-b-D-threo-pentofuranosyl)adenine, an acid stable purine-based reverse transcriptase inhibitor discovered by the NIH and under development by U.S. Bioscience Inc., West Conshohoken, Pa. 19428.

The term “non-nucleoside reverse transcriptase inhibitors” (“NNRTI”s) as used herein means non-nucleosides that inhibit the activity of HIV-1 reverse transcriptase. Typical suitable NNRTIs include nevirapine (BI-RG-587) available under the VIRAMUNE tradename from Boehringer Ingelheim, the manufacturer for Roxane Laboratories, Columbus, Ohio 43216; delaviradine (BHAP, U-90152) available under the RESCRIPTOR tradename from Pharmacia & Upjohn Co., Bridgewater N.J. 08807; efavirenz (DMP-266) a benzoxazin-2-one disclosed in WO94/03440 and available under the SUSTIVA tradename from DuPont Pharmaceutical Co., Wilmington, Del. 19880-0723; PNU-142721, a furopyridine-thio-pyrimide under development by Pharmacia and Upjohn, Bridgewater N.J. 08807; AG-1549 (formerly Shionogi #S-1153); 5-(3,5-dichlorophenyl)-thio-4-isopropyl-1-(4-pyridyl)methyl-I H-imidazol-2-ylmethyl carbonate disclosed in WO 96/10019 and under clinical development by Agouron Pharmaceuticals, Inc., La Jolla Calif. 92037-1020; MKC442 (1-(ethoxymethyl)-5-(1-methylethyl)-6-(phenylmethyl)-(2,4(1H,3H)-pyrimidi-nedione) discovered by Mitsubishi Chemical Co. and under development by Triangle Pharmaceuticals, Durham, N.C. 27707; and (+)-calanolide A (NSC-675451) and B, coumarin derivatives disclosed in NIH U.S. Pat. No. 5,489,697, licensed to Med Chem Research, which is co-developing (+) calanolide A with Vita-Invest as an orally administrable product.

The term “protease inhibitor” (“PI”) as used herein means inhibitors of the HIV-1 protease, an enzyme required for the proteolytic cleavage of viral polyprotein precursors (e.g., viral GAG and GAG Pol polyproteins), into the individual functional proteins found in infectious HIV-1. HIV protease inhibitors include compounds having a peptidomimetic structure, high molecular weight (7600 daltons) and substantial peptide character, e.g. CRIXIVAN (available from Merck) as well as nonpeptide protease inhibitors e.g., VIRACEPT (available from Agouron). Typical suitable PIs include saquinavir (Ro 31-8959) available in hard gel capsules under the INVIRASE tradename and as soft gel capsules under the FORTOVASE tradename from Roche Pharmaceuticals, Nutley, N.J. 07110-1199; ritonavir (ABT-538) available under the NORVIR tradename from Abbott Laboratories, Abbott Park, Ill. 60064; indinavir (MK-639) available under the CRIXIVAN tradename from Merck & Co., Inc., West Point, Pa. 19486-0004; nelfnavir (AG-1343) available under the VIRACEPT tradename from Agouron Pharmaceuticals, Inc., La Jolla Calif. 92037-1020; amprenavir (141W94), tradename AGENERASE, a non-peptide protease inhibitor under development by Vertex Pharmaceuticals, Inc., Cambridge, Mass. 02139-4211 and available from Glaxo-Wellcome, Research Triangle, N.C. under an expanded access program; lasinavir

(BMS-234475) available from Bristol-Myers Squibb, Princeton, N.J. 08543 (originally discovered by Novartis, Basel, Switzerland (CGP-61755); DMP-450, a cyclic urea discovered by Dupont and under development by Triangle Pharmaceuticals; BMS-2322623, an azapeptide under development by Bristol-Myers Squibb, Princeton, N.J. 08543, as a 2nd-generation HIV-1 PI; ABT-378 under development by Abbott, Abbott Park, Ill. 60064; and AG-1549 an orally active imidazole carbamate discovered by Shionogi (Shionogi #S-1153) and under development by Agouron Pharmaceuticals, Inc., La Jolla Calif. 92037-1020.

Other antiviral agents include hydroxyurea, ribavirin, IL-2, IL-12, pentafuside and Yissum Project No. 11607. Hydroxyurea (Droxia), a ribonucleoside triphosphate reductase inhibitor, the enzyme involved in the activation of T-cells, was discovered at the NCI and is under development by Bristol-Myers Squibb; in preclinical studies, it was shown to have a synergistic effect on the activity of didanosine and has been studied with stavudine. IL-2 is disclosed in Ajinomoto EP-0142268, Takeda EP-0176299, and Chiron U.S. Pat. Nos. RE 33,653, 4,530,787, 4,569,790, 4,604,377, 4,748,234, 4,752,585, and 4,949,314, and is available under the PROLEUKIN (aldesleukin) tradename from Chiron Corp., Emeryville, Calif. 94608-2997 as a lyophilized powder for IV infusion or sc administration upon reconstitution and dilution with water; a dose of about 1 to about 20 million IU/day, sc is preferred; a dose of about 15 million IU/day, sc is more preferred. IL-12 is disclosed in WO96/25171 and is available from Roche Pharmaceuticals, Nutley, N.J. 07110-1199 and American Home Prodocts, Madison, N.J. 07940; a dose of about 0.5 microgram/kg/day to about 10 microgram/kg/day, sc is preferred. Pentafuside (DP-178, T-20) a 36-amino acid synthetic peptide, is disclosed in U.S. Pat. No. 5,464,933 licensed from Duke University to Trimeris which is developing pentafuside in collaboration with Duke University; pentafuside acts by inhibiting fusion of HIV-1 to target membranes. Pentafuside (3 100 mg/day) is given as a continuous sc infusion or injection together with efavirenz and 2 PI's to HIV-1 positive patients refractory to a triple combination therapy; use of 100 mg/day is preferred. Yissum Project No. 11607, a synthetic protein based on the HIV-1 Vif protein, is under preclinical development by Yissum Research Development Co., Jerusalem 91042, Israel. Ribavirin, 1-.beta.-D-ribofuranosyl-1H-1,2,4-triazole-3-carboxamide, is available from ICN Pharmaceuticals, Inc., Costa Mesa, Calif; its manufacture and formulation are described in U.S. Pat. No. 4,211,771.

The term “derivative” of a compound, as used herein, refers to a chemically modified compound wherein the chemical modification takes place either at a functional group of the compound or on the aromatic ring.

As used herein, the term “subject” refers to organisms to be treated by the methods and compounds of the present invention. Such organisms preferably include, but are not limited to, mammals (e.g., murines, simians, equines, bovines, porcines, canines, felines, and the like), and most preferably includes humans. In the context of the invention, the term “subject” generally refers to an individual who will receive or who has received treatment (e.g., administration of a compound of the present invention and optionally one or more other agents) for a condition characterized by HIV infection.

As used herein, the term “host cell” refers to any eukaryotic or prokaryotic cell (e.g., mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

In some embodiments, the “target cells” of the compositions and methods of the present invention include, refer to, but are not limited to, lymphoid cells or cancer cells. Lymphoid cells include B cells, T cells, and granulocytes. Granulocyctes include eosinophils and macrophages. In some embodiments, target cells are continuously cultured cells or uncultered cells obtained from patient biopsies.

As used herein, the term “co-administration” refers to the administration of at least two agent(s) (e.g., a compound of the present invention) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “toxic” refers to any detrimental or harmful effects on a cell or tissue as compared to the same cell or tissue prior to the administration of the toxicant.

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁻, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “pathogen” refers a biological agent that causes a disease state (e.g., infection, AIDS, etc.) in a host. “Pathogens” include, but are not limited to, viruses (e.g., HIV), bacteria, archaea, fungi, protozoans, mycoplasma, prions, and parasitic organisms.

As used herein, the term “virus” refers to minute infectious agents, which with certain exceptions, are not observable by light microscopy, lack independent metabolism, and are able to replicate only within a living host cell. The individual particles (i.e., virions) typically consist of nucleic acid and a protein shell or coat; some virions also have a lipid containing membrane. The term “virus” encompasses all types of viruses, including animal, plant, phage, and other viruses. An example of a virus includes, but is not limited to, HIV.

As used herein, the terms “purified” or “to purify” refer, to the removal of undesired components from a sample. As used herein, the term “substantially purified” refers to molecules that are at least 60% free, preferably 75% free, and most preferably 90%, or more, free from other components with which they usually associated.

As used herein, the terms “non-specific binding” and “background binding” when used in reference to the interaction of a molecule with a protein or enzyme refers to an interaction that is not dependent on the presence of a particular structure.

As used herein, the term “modulate” refers to the activity of a compound (e.g., a compound of the present invention) to affect (e.g., to promote or retard) an aspect of cellular function, including, but not limited to, enzymatic activity, maturation, cell growth, replication, proliferation, and the like.

The term “test compound” refers to any chemical entity, pharmaceutical, drug, and the like, that can be used to treat or prevent a disease, illness, sickness, or disorder of bodily function, or otherwise alter the physiological or cellular status of a sample (e.g., the level of dysregulation of apoptosis in a cell or tissue). Test compounds comprise both known and potential therapeutic compounds. A test compound can be determined to be therapeutic by using the screening methods of the present invention. A “known therapeutic compound” refers to a therapeutic compound that has been shown (e.g., through animal trials or prior experience with administration to humans) to be effective in such treatment or prevention.

### DETAILED DESCRIPTION OF THE INVENTION

The present invention relates to HIV protease, and methods for inhibiting the function of HIV protease. In particular, present invention provides compounds that inhibit or block the biological activity of HIVp, thereby causing the replication of the HIV virus to be inhibited or to terminate. These compounds, as well as pharmaceutical compositions that contain these compounds and optionally other anti-viral agents as active ingredients, are suitable for treating patients or hosts infected with the HIV virus, which is known to cause AIDS. The compounds and formulations also find use in diagnostic and research settings.

Exemplary compositions and methods of the present invention are described in more detail in the following sections: I. A Novel HIV Protease Binding Site; II. Exemplary Compounds; III. Pharmaceutical Compositions, Formulations, and Exemplary Administration Routes and Dosing Considerations; IV. Drug Screens; V. Therapeutic Applications; and VI. Diagnostic Applications.

The practice of the present invention employs, unless otherwise indicated, conventional techniques of organic chemistry, pharmacology, molecular biology (including recombinant techniques), cell biology, biochemistry, and immunology, which are within the skill of the art. Such techniques are explained fully in the literature, such as, “Molecular cloning: a laboratory manual” Second Edition (Sambrook et al., 1989); “Oligonucleotide synthesis” (M. J. Gait, ed., 1984); “Animal cell culture” (R. I. Freshney, ed., 1987); the series “Methods in enzymology” (Academic Press, Inc.); “Handbook of experimental immunology” (D. M. Weir & C. C. Blackwell, eds.); “Gene transfer vectors for mammalian cells” (J. M. Miller & M. P. Calos, eds., 1987); “Current protocols in molecular biology” (F. M. Ausubel et al., eds., 1987, and periodic updates); “PCR: the polymerase chain reaction” (Mullis et al., eds., 1994); and “Current protocols in immunology” (J. E. Coligan et al., eds., 1991), each of which is herein incorporated by reference in its entirety.

### I. A Novel HIV Protease Binding Site

HIV protease (HIVp) is an aspartyl protease that is essential for the life-cycle of HIV. It cleaves the envelope polyprotein gp160 into envelope glycoproteins gp120 and gp41 in the golgi apparatus in the final stages of the HIV life-cycle. Thus, inhibition of this protease prevents maturation of HIV particles. As such, many drugs have been developed, so-called protease inhibitors, that target this enzyme (see, e.g., Brik A, et al., (2003) Org. Biomol. Chem. 1 (1): 5-14; herein incorporated by reference in its entirety). Because HIV is a retrovirus, the active site of this enzyme will change occasionally due to mutations. To combat this, a cocktail of drugs is often used rather than a single one.

In experiments conducted during the development of embodiments for the present invention, molecular dynamics (MD) simulations of three forms of apo HIVp based on the crystal structures of 1HHP, 3HVP, and 3PVH were conducted. Images were taken from those MD simulations to provide an ensemble of conformational states of the open, unbound form of HIVp. These images were subjected to a computational solvent-mapping technique. The images were then overlaid to identify “regions of consensus” or sites on the protein surfaces that consistently mapped out favorable interactions within a localized space (see, e.g., Meagher, et al., (2004) J. Am. Chem. Soc. 126:13276-13281; Meagher, et al., (2006) J. Med. Chem. 49:3478-3484; each herein incorporated by reference in their entireties).

Structure-based drug design (SBDD) is almost always based on crystal structures of proteins with bound inhibitors. Within the field, it is considered nearly impossible to successfully conduct SBDD with an unbound structure of a binding site. For over 20 years, the field has pursued inhibitors of HIVp, based on bound structures in the closed state. In experiments conducted during the development of embodiments for the present invention, the semi-open and open states of HIVp were investigated with SBDD. Such experiments led to the discovery of a novel targeting region of HIVp defined by a lower portion (e.g., defined by I84, V32, P81, T80, P79, and G78), an upper portion (e.g., defined by V56, I54, I47, G48, G49, and I50), and distal contacts (e.g., defined by V82 and the backbone atoms of V77, L33, and K55). The newly identified HIVp binding site is only present in the more open states.

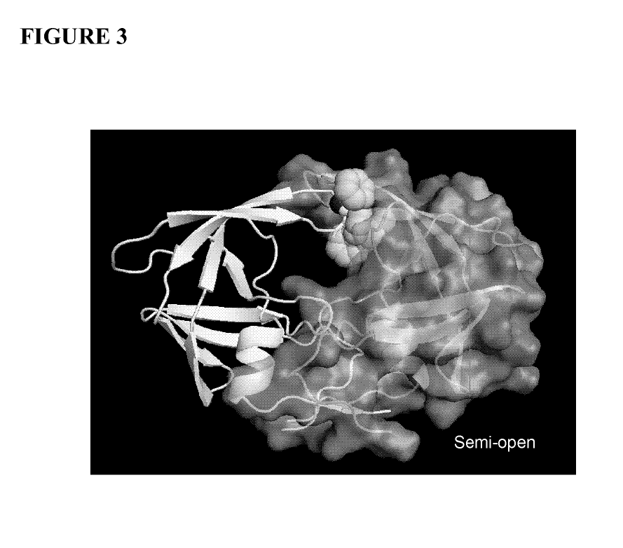

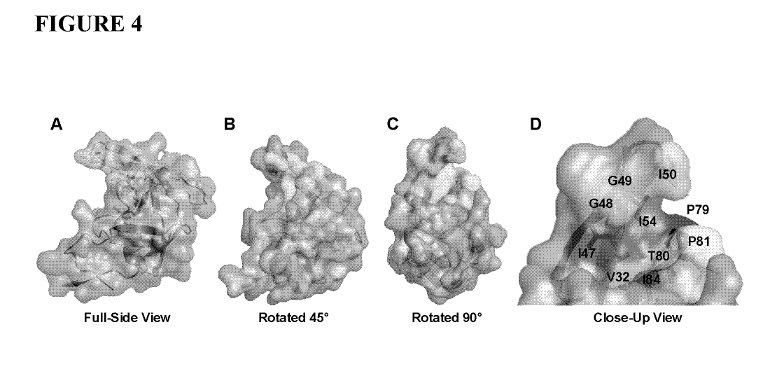

The newly identified HIVp binding site is shown in FIG. 3. The newly identified HIVp binding site represents an attractive target for modulating HIVp function (e.g., inhibiting HIVp enzyme activity, inhibiting maturation of HIV particles, terminating replication of the HIV virus) as it is essential in forming the closed conformer of HIVp. For example, when a dimer folds in the closed state, each monomer places its flap tips against the other monomer, within the newly identified HIVp binding site. If the flaps cannot close and coordinate the central water molecule, the catalytic efficiently of HIVp drops. In addition, some of the residues within the newly identified HIVp binding site are highly conserved and may be resistant to giving rise to escape mutants. In addition, the newly identified HIVp binding site is much smaller than the central cavity suggesting that inhibitors of this site have low molecular weights which could have better pharmacokinetic properties than current HIVp drugs. Moreover, existing inhibitors could be modified to contain additional groups that target the newly identified HIVp binding site. FIG. 4 shows a molecular image of a semi-open state of HIVp, and that the upper portion of the newly identified HIVp binding site is defined by V56, I54, I47, G48, G49, and 150, the lower portion defined by I84, V32, P81, T80, P79, and G78, and the distal contacts defined by V82 and the backbone atoms of V77, L33, and K55.

In certain embodiments, the present invention provides methods for treating HIV and HIV related disorders (e.g., AIDS, AIDS Related Complex, HIV Infection, and associated complications and symptoms) through targeting of the newly identified HIVp binding site by thereby modulating the function of HIVp (e g., inhibiting HIVp enzyme activity, inhibiting maturation of HIV particles, terminating replication of the HIV virus). The present invention is not limited to a particular manner of modulating the function of HIVp through targeting of the newly identified HIVp binding site. In some embodiments, the present invention provides compounds capable of targeting the newly identified HIVp binding site. In some embodiments, the present invention provides methods for treating HIV and HIV related disorders through administering drugs known to inhibit HIVp (e.g., protease inhibitors) and drugs capable of binding the newly identified HIVp binding site.

In certain embodiments, the present invention provides methods for screening drugs through identifying compounds capable of, for example, binding with the newly identified HIVp binding site. In some embodiments, the present invention provides methods for screening drugs capable of inhibiting the function of HIVp (e g., inhibiting HIVp enzyme activity, inhibiting maturation of HIV particles, terminating replication of the HIV virus) through targeting of the newly identified HIVp binding site.

### II. Exemplary Compounds

In certain embodiments, the present invention provides compounds able to inhibit HIV protease (HIVp) function (e g., inhibiting HIVp enzyme activity, inhibiting maturation of HIV particles, terminating replication of the HIV virus). The compounds are not limited to a particular manner of inhibiting HIVp function. In some embodiments, the compounds inhibit HIVp function through binding with HIVp. The compounds of the present invention are not limited to a particular manner of binding with HIVp. In experiments conducted during the course of development of embodiments for the present invention, a novel targeting region of HIVp was identified defined by a lower portion (e.g., defined by I84, V32, P81, T80, P79, and G78), an upper portion (e.g., defined by V56, I54, I47, G48, G49, and I50), and distal contacts (e.g., defined by V82 and the backbone atoms of V77, L33, and K55). In some embodiments, the compounds inhibit HIVp through targeting only the upper portion of the newly identified HIVp binding site. In some embodiments, the compounds inhibit HIVp through targeting only the lower portion of the newly identified HIVp binding site. In some embodiments, the compounds inhibit HIVp through targeting one more portions of the upper portion, lower portion and/or distal contacts of the newly identified HIVp binding site. In some embodiments, the compounds inhibit HIVp through targeting the upper and lower portions of the newly identified HIVp binding site. In some embodiments, the compounds inhibit HIVp through targeting the upper portion and the distal contacts of the newly identified HIVp binding site. In some embodiments, the compounds inhibit HIVp through targeting the lower portion and the distal contacts of the newly identified HIVp binding site. In some embodiments, the compounds inhibit HIVp through targeting the upper portion, the lower portion and the distal contacts of the newly identified HIVp binding site.

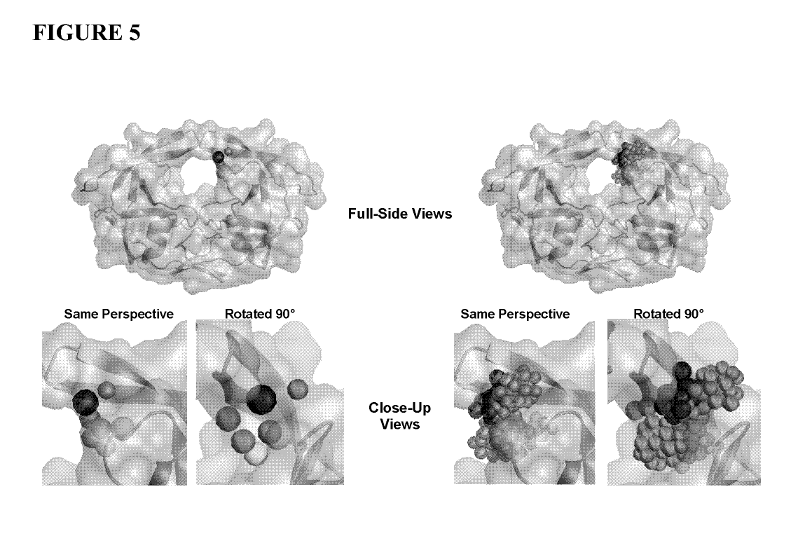

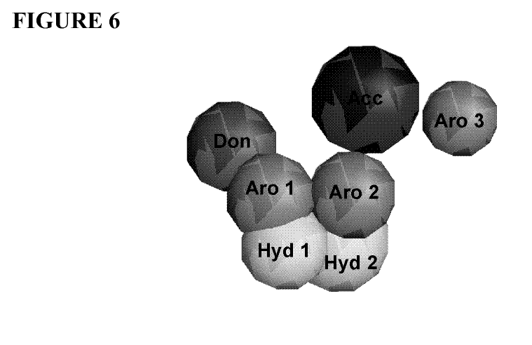

The present invention is not limited to certain types or kinds of compounds. In some embodiments, the compounds are able to bind the newly identified HIVp binding site. FIGS. 5 and 6 depict pharmacophores models of molecules able to bind the newly identified HIVp binding site. In some embodiments, the compounds able to bind the newly identified HIVp binding site are modeled after the pharmacophores depicted in FIGS. 5 and 6. The pharmacophore models in FIGS. 5 and 6 and in Tables 1 and 2 describe the chemical features of molecules that complement the newly identified HIVp binding. The Cartesian coordinates for the various chemical functionalities are given relative to the 1HHP crystal structure; they complement the location of the newly identified HIVp binding at the base of the flexible flap in a monomer of HIV protease. The last column gives the tolerance for the location of a complementing functional group (e.g., the centroid of an aromatic group must be placed within 1.592 Ångstroms of the coordinates noted on the first line). The tolerance was determined by multiplying the RMSD of the solvent-mapping probes by 1.3 (tighter tolerance) and 1.5 (looser tolerance). Solvent mapping of the protein surface was the computational technique used to identify the new site.

TABLE 1
The Pharmacophore Sites Described in Cartesian Coordinates
and Tight vs Loose Tolerances (in Ångstroms)

| Chemical | | | | | Tolerance |
|---|---|---|---|---|---|
| Features | X | Y | Z | Tolerance (1.3) | (1.5) |
| Aromatic 1 | 37.223 | 38.637 | −5.538 | 1.592 | 1.837 |
| Aromatic 2 | 34.737 | 39.215 | −6.802 | 1.582 | 1.825 |
| Aromatic 3 | 30.129 | 37.435 | −7.590 | 1.367 | 1.577 |
| Hydrophobic 1 | 37.650 | 39.514 | −7.048 | 1.569 | 1.810 |
| Hydrophobic 2 | 35.959 | 38.190 | −9.007 | 1.834 | 2.116 |
| Donor | 37.526 | 35.713 | −4.966 | 1.636 | 1.888 |
| Acceptor | 32.651 | 37.225 | −4.985 | 1.963 | 2.265 |

Table 2 describes the pharmacophores in terms of distances, rather than Cartesian coordinates:

TABLE 2
Distances (in Ångstroms) between the Pharmacophore Sites

| | Aro 1 | Aro 2 | Aro 3 | Hyd 1 | Hyd 2 | Don | Acc |
|---|---|---|---|---|---|---|---|
| Aromatic 1 | 0.000 | 2.848 | 7.482 | 1.798 | 3.719 | 2.995 | 4.817 |
| Aromatic 2 | | 0.000 | 5.002 | 2.939 | 2.721 | 4.839 | 3.408 |
| Aromatic 3 | | | 0.000 | 7.822 | 6.047 | 8.035 | 3.632 |
| Hydrophobic 1 | | | | 0.000 | 2.907 | 4.336 | 5.872 |
| Hydrophobic 2 | | | | | 0.000 | 4.992 | 5.296 |
| Donor | | | | | | 0.000 | 5.104 |
| Acceptor | | | | | | | 0.000 |

The pharmacophore model described in FIG. 6 was used to identify several compounds from a set of 35,000 compounds. In the virtual screen, each hit was required to contain at least 6 of the 7 pharmacophore sites shown in the model. 93 compounds were identified with a molecular weight of ≦360. Visual inspection was used to select the compound class with the best agreement to the model, and the following molecules were identified for testing:

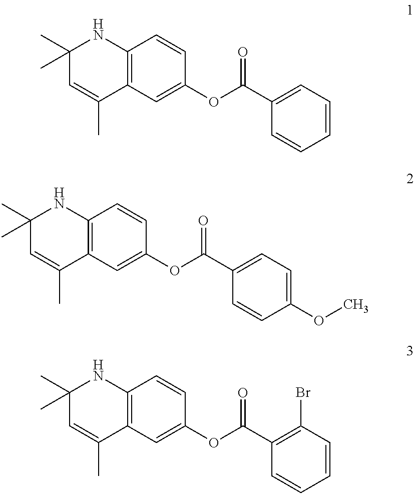

Compound 1 was auto-fluorescent and could not be evaluated by the assay. Compound 2 has show an IC₅₀ of 18 μM. Compound 3 has a bromine in the ortho position of the terminal phenyl ring (instead of a para-methoxy substitution seen in compound 2); it was also selected for testing. A compound with a chlorine in the ortho position had been identified in the virtual screen mentioned in the preceding paragraph, but the bromo was the compound that was readily available on-site for testing. This compound was shown to be active. Thus, both compounds tested using the criteria above were shown to inhibit HIVp through, for example, binding to the newly identified site. MD simulations confirmed the stability of this inhibitor class within the new site and the inability of the flaps to adopt a closed, “reactive” form in their presence. These inhibitors are much smaller than existing protease inhibitors and chemically very distinct. There is little likelihood that they are acting as traditional competitive inhibitors within the enzymatic binding site of HIVp. Modeling has indicated that these complexes are not stable within the central pocket; instead, the complexes migrate to the new site and form appropriate complexes.

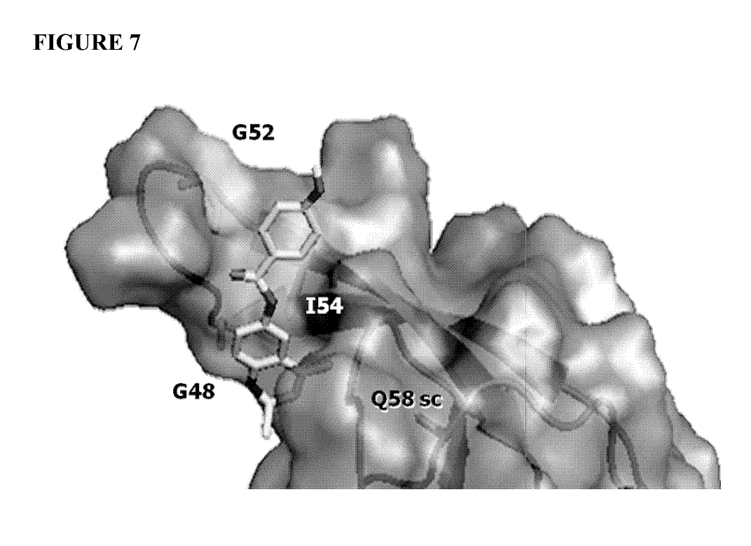

The complex of HIVp with compound 2 was examined using 1H-15N HSQC spectra from NMR. Spectra were obtained in the presence of excess concentrations of the inhibitor as well as in its absence. Only four shifts were observed: amides of G48, G52, I54, and the side chain of Q58 (FIGS. 7). G52, G48, and I54 were central to the new site, particularly I54. In addition, the absence of shifts within the standard residues at the bottom of the pocket further support the new mode of binding.

A search for similar compounds was conducted using the ZINC database (www, followed by, .ZINC.docking org). In some embodiments, compounds able to bind the newly identified HIVp binding site comprise the followings seven characteristics: three aromatic groups, two hydrophobic groups, one hydrogen bond donor group, and one hydrogen bond acceptor group. Based on the similar compounds, compounds able to bind the newly identified HIVp binding site are described by the following formula: C1C2-A-B, including salts, esters, and prodrugs thereof, and including both R and S enantiomeric forms and racemic mixtures thereof. In some embodiments, the compound comprises three aromatic groups, one at position C1, one at position C2, one at position B, two hydrophobic groups, one hydrogen bond donor group, and one hydrogen bond acceptor group, wherein A provides a covalent linker connecting C1C2 and B. In some embodiments, the C1 group is not aromatic. In some embodiments, the position of groups is defined by ranges of distances and positions with respect to one another as described in Tables 1 and 2. In some embodiments wherein the compound is described by the formula: C1C2-A-B, the compound has at least six of the seven characteristics of the newly identified HIVp binding site (e.g., an aromatic group at position C1, an aromatic group at position C2, an aromatic group at position B, two hydrophobic groups, one hydrogen bond donor group, and one hydrogen bond acceptor group). In some embodiments, the compound described by the formula C1C2-A-B has all seven of these characteristics. In some embodiments, the compound described by the formula C1C2-A-B has six of the seven characteristics. In some embodiment where the compound described by C1C2-A-B has only six of the seven characteristics, the C1 group is not aromatic.

In some embodiments where the compound is described by the formula C1C2-A-B, “C1C2” includes, but is not limited to, the following structures:

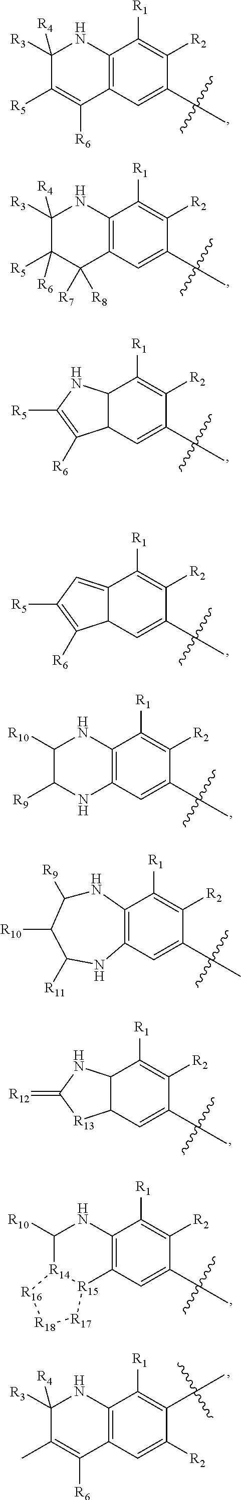

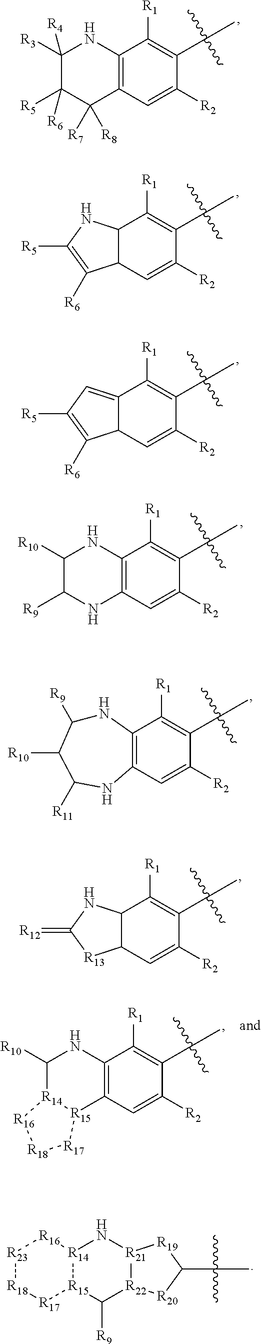

In some embodiments, R1 and R2 are independently hydrogen, hydrogen-bond acceptors or donors (e.g., OH). In some embodiments, R3, R4, R5, R6, R7 and R8 can be independently functionalized with small hydrophobic groups (e.g., methyl, ethyl, iodo, bromo, and chloro—R5, R6, R7 or R8 can also have a small hydrophilic group such as hydroxyl). In some embodiments, R3 and R4 are each methyl, and R5 and R6 are each hydrogen. In some embodiments, R9, R10 and R11 are independently halogen (e.g., Cl, I, Br, F), C-(halogen (e.g., Cl, I, Br, F))₃, CF₃, CI₃, CBr₃, CCl₃, COO⁻, COOH, ketone, hydrogen and methyl. In some embodiments, R12 and R13 is independently O or S. In some embodiments, R14 and 15 is either C═C or CH—CH. In some embodiments, R16, R17, R18, and R23 are independently present or absent, and if present are independently COO⁻, COOH, ketone, hydrogen, halogen (e.g., Cl, I, Br, F), C-(halogen (e.g., Cl, I, Br, F))₃, CF₃, CI₃, CBr₃, CCl₃, C, CH, CH2, and methyl. In some embodiments, R19 and R20 are independently C, CH, CH2, N, and NH. In some embodiments, R21 and R22 is C, CH and N.

In some embodiments, C1C2 is described by the following structures:

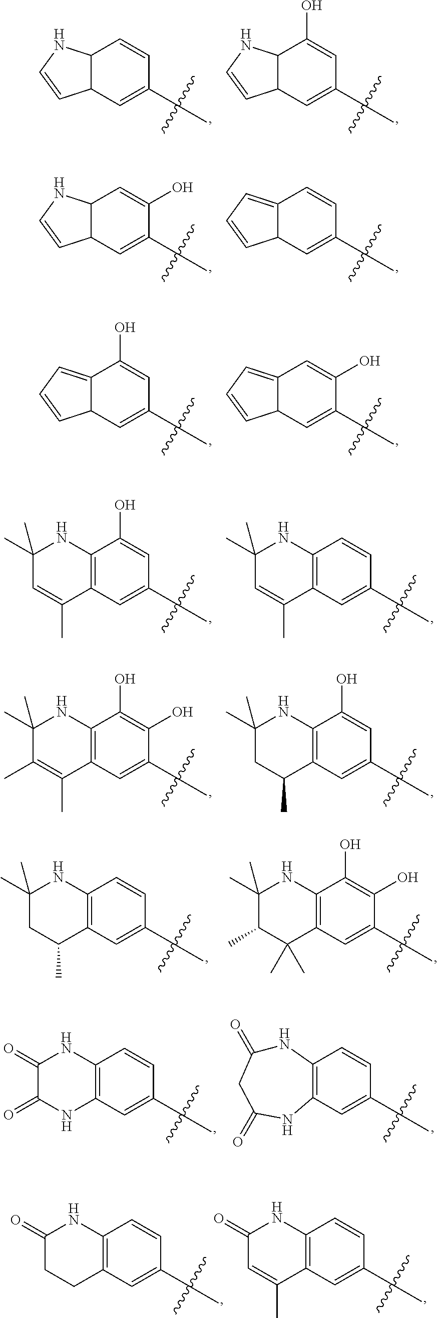

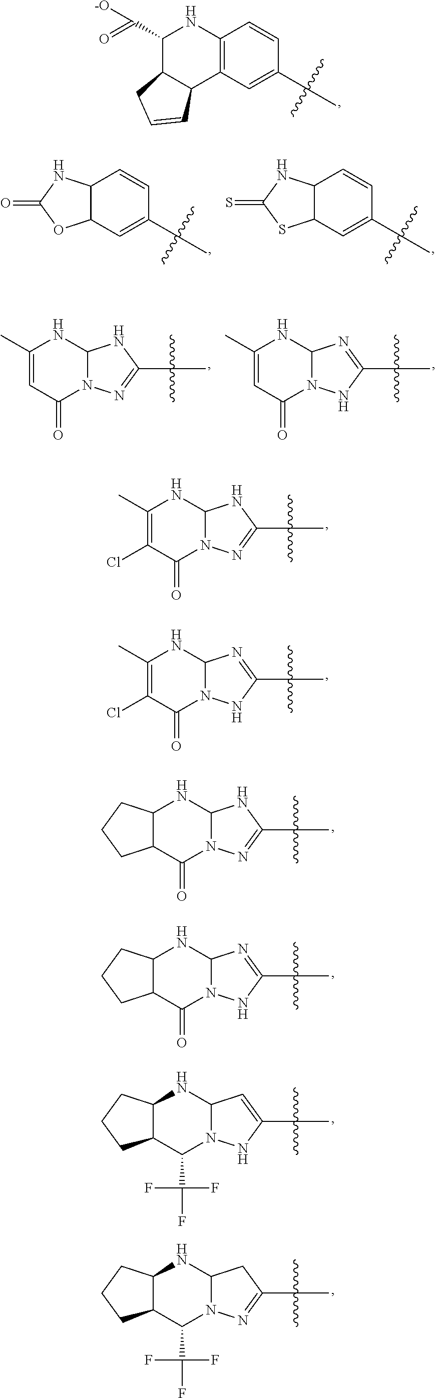

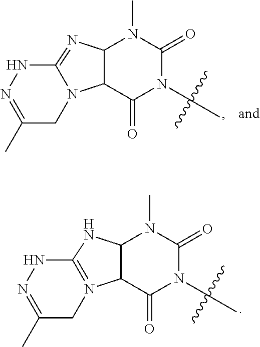

In some embodiments, “A” is a covalent linker connecting “C1C2” and “B.” In some embodiments, examples of “A” include, but are not limited to, chemical moieties comprising esters, ethers, amides, amines, ketones, thioesters, thioethers, thioamides, thioketones and short alkyl chains. In some embodiments, “A” is covalently attached to any part of “C1C2” and “B.” In some embodiments, “A” is attached to the R2 portion of the following “C1C2” formulas: In some embodiments, “A” includes, but is not limited to, the following structures:

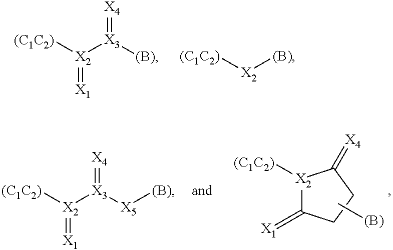

where X1 and X4 are present or absent and, if present, are independently selected from O and S, and where X2, X3 and X5 are independently selected from O, C, S, SO₂, PO₂, N, or NH.

In some embodiments, examples of “A” include, but are not limited to, chemical moieties comprising sulfonates, sulfonamides, phosphonates, and/or phosphamides. In some embodiments, use of one or more phosphate groups comprising ³¹P and/or ³²P within “A” permits improved labeling for detection purposes (e.g., NMR detection; scintillation detection). In some embodiments, other detectable atoms may be incorporated within the compound for detection and/or labeling purposes. In some embodiments, “A” is a phosphate linkage. In some embodiments, “A” is a phosphate linkage. In some embodiments, “A” is a sulfate linkage. In some embodiments, “A” includes, but is not limited to, the following structures:

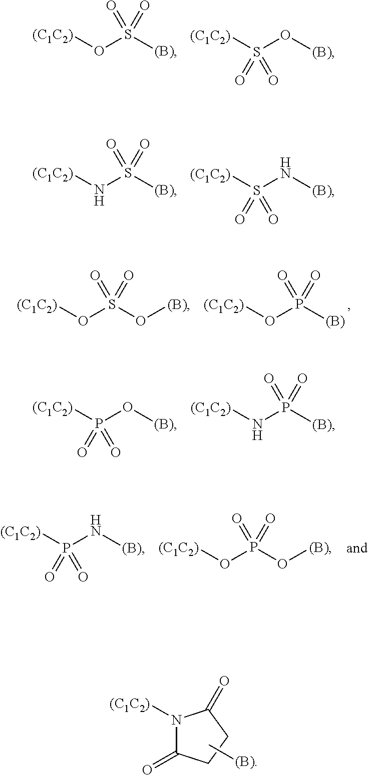

In some embodiments where the compound is described by the formula C1C2-A-B, “B” includes, but is not limited to, the following structures:

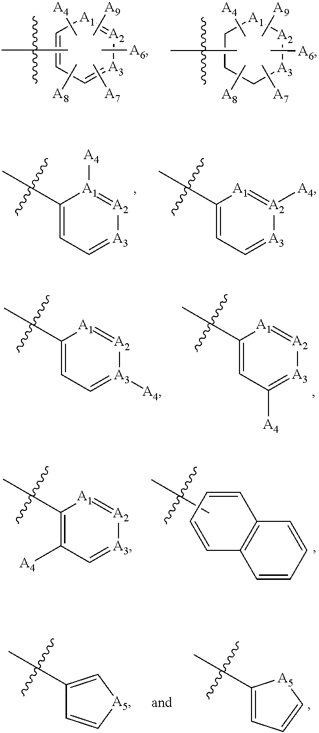

where A1, A2, and A3 are independently selected from C and N, where A4, A6, A7, A8, and A9 are independently present or absent, and if present, are independently halogen (e.g., Cl, I, Br, F), C-(halogen (e.g., Cl, I, Br, F))₃, CF₃, CI₃, CBr₃, CCl₃,

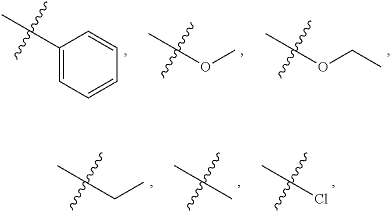

or a solubilizing group

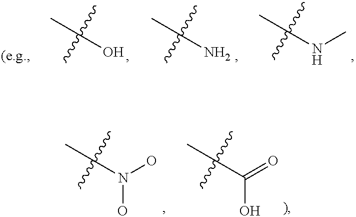

and where A5 is selected from O, C, S, or NH. In some embodiments, examples of “B” include, but are not limited to.

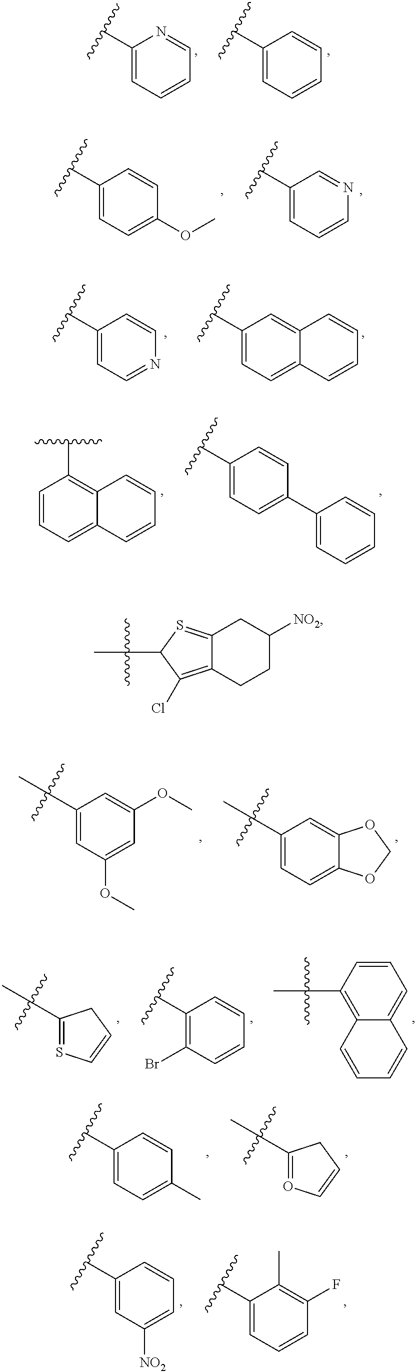

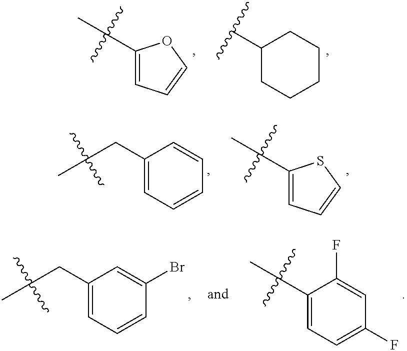

In some embodiments, “B” is a non-aromatic cyclical compound. Examples include, but are not limited to,

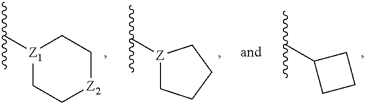

where Z, Z1 and Z2 are independently C or N. In some embodiments, Z1 and Z2 are the same. In some embodiments, Z1 and Z2 are different. In some embodiments, B is an aromatic ring. In some embodiments, the aromatic ring is benzene.

In some embodiments, B is an aromatic ring substituted at the ortho, meta, and/or para positions either singly or in combination. The B subgroup is not limited to a particular type of substitution group. In some embodiments, the B subgroup is an aromatic ring substituted with one or more of the following chemical moieties: hydroxyl, methoxyl, ethoxyl, methyl, ethyl, isopropyl, amine, methylamine, and halides. In some embodiments, larger substitution groups are included at the para position of the aromatic group. In some embodiments, the solubility of the compound is improved by having a hydrophilic (e.g., OH) group positioned at the para position of the aromatic ring.

In some embodiments, C1C2-A-B is described by any of the following formulas:

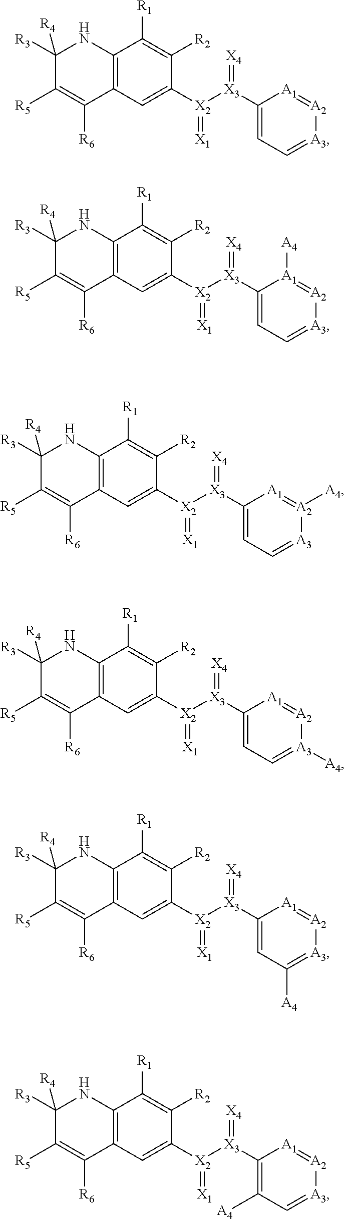

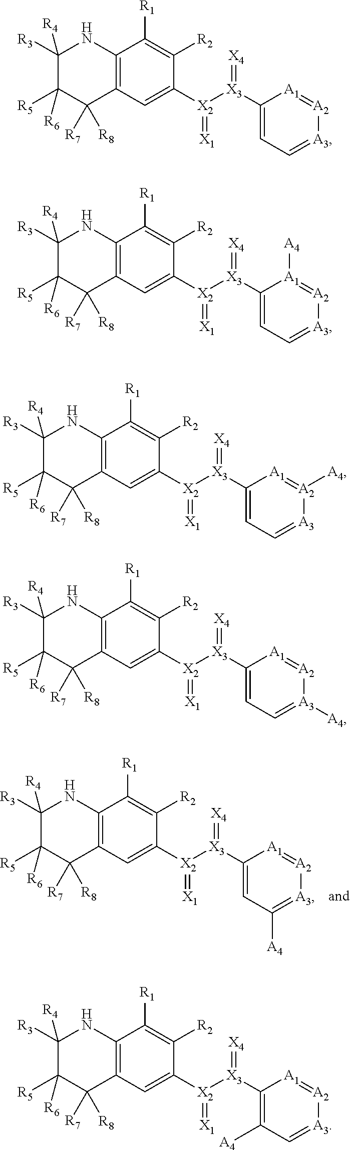

In certain embodiments, the exemplary compounds of the formula C1C2-A-B are provided in Table 3, R1 and R2 are independently hydrogen, hydrogen-bond acceptors or donors (e.g., OH), R3, R4, R5, R6, R7 and R8 can be independently functionalized with small hydrophobic groups (e.g., methyl, ethyl, iodo, bromo, and chloro—R5, R6, R7 or R8 can also have a small hydrophilic group such as hydroxyl), where R9, R10 and R11 are independently halogen (e.g., Cl, I, Br, F), C-(halogen (e.g., Cl, I, Br, F))₃, CF₃, CI₃, CBr₃, CCl₃, COO⁻, COOH, ketone, hydrogen and methyl, R12 and R13 is independently O or S, where R14 and 15 is either C═C or CH—CH, R16, R17, R18, and R23 are independently present or absent, and if present are independently COO⁻, COOH, ketone, hydrogen, halogen (e.g., Cl, I, Br, F), C-(halogen (e.g., Cl, I, Br, F))₃, CF₃, CI₃, CBr₃, CCl₃, C, CH, CH2, and methyl, where R19 and R20 are independently C, CH, CH2, N, and NH, where R21 and R22 is C, CH and N, where X1 and X4 are present or absent and, if present, are independently selected from O and S, where X2, X3 and X5 are independently selected from O, C, S, SO₂, PO₂, N, or NH, where A1, A2, and A3 are independently selected from C and N, where A4, A6, A7, A8, and A9 are independently present or absent, and if present, are independently halogen (e.g., Cl, I, Br, F), C-(halogen (e.g., Cl, I, Br, F))₃, CF₃, CI₃, CBr₃, CCl₃,

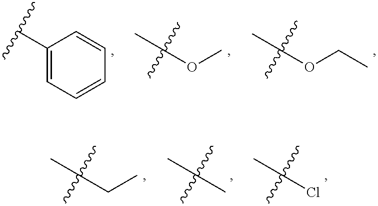

or a solubilizing group

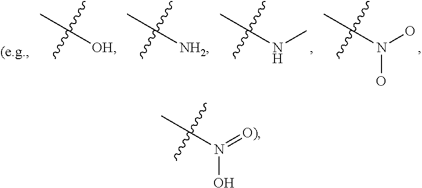

and where A5 is selected from O, C, S, or NH.

TABLE 3

| C1C2 | A | B |
|---|---|---|
| 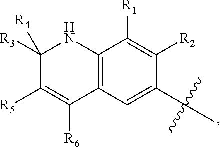 | 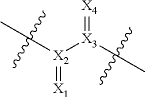 | 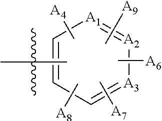 |
| 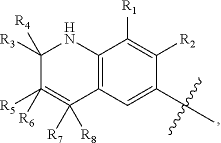 | | 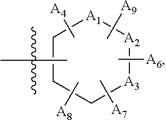 |
| 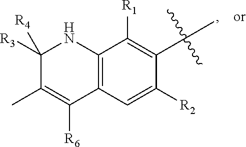 | | 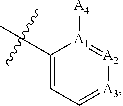 |
| 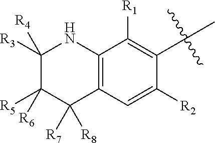 | | 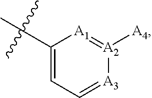 |
| | | 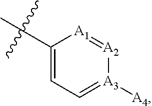 |
| | | 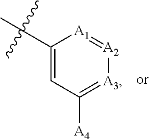 |
| | | 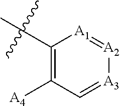 |
| 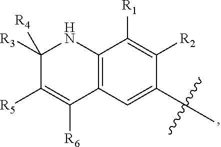 | 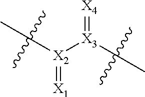 | 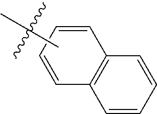 |
| 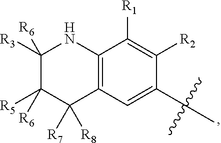 | | |
| 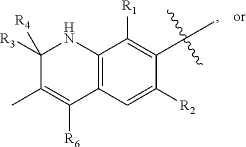 | | |
| 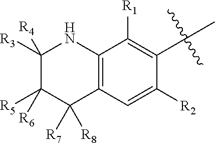 | | |
| 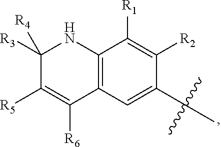 | 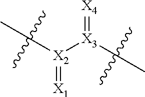 | 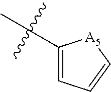 |
| 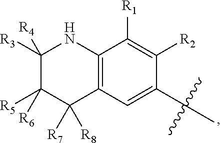 | | |
| 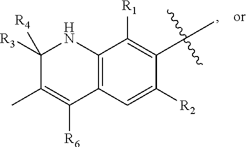 | | |
| 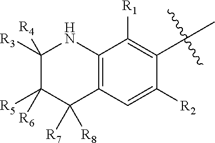 | | |
| 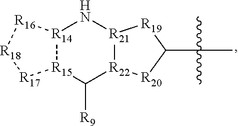 | 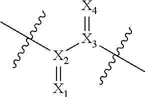 | 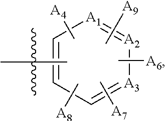 |
| 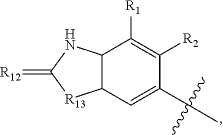 | | 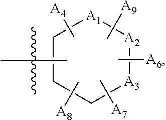 |
| 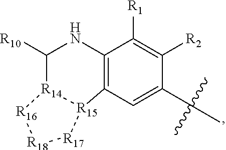 | | 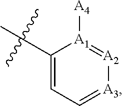 |
| 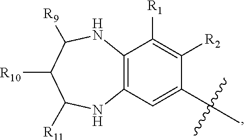 | | 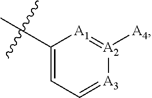 |
| 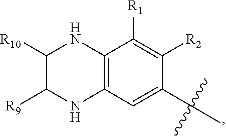 | | 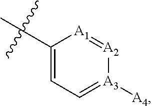 |
| 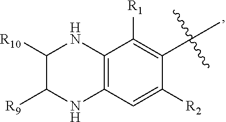 | | 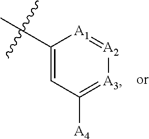 |
| 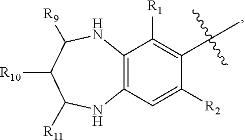 | | 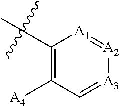 |
| 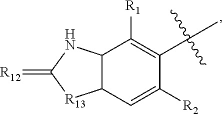 | | |
| 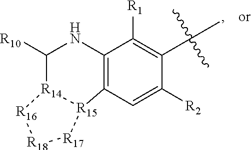 | | |
| 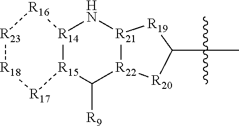 | | |
| 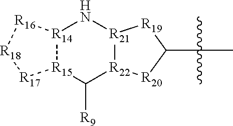 | 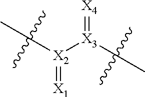 | 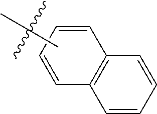 |
| 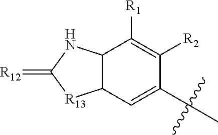 | 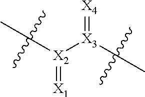 | 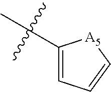 |
| 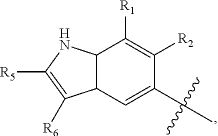 | 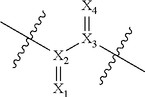 | 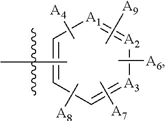 |
| 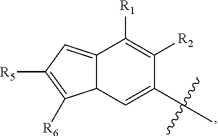 | | 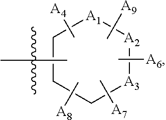 |
| 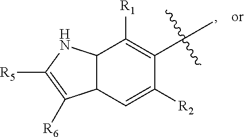 | | 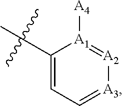 |
| 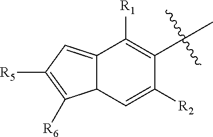 | | 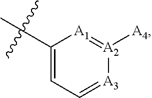 |
| | | 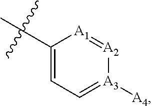 |
| | | 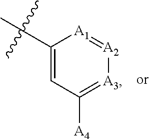 |
| | | 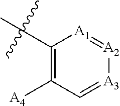 |
| 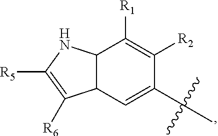 | 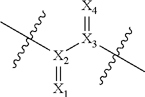 | 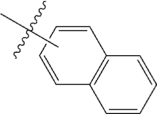 |
| 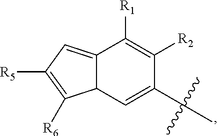 | | |
| 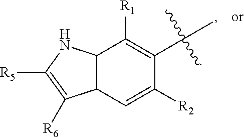 | | |
| 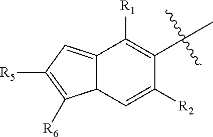 | | |
| 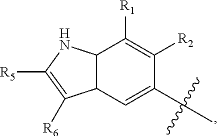 | 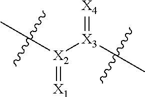 | 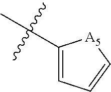 |
| 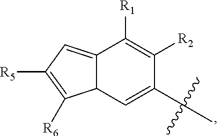 | | |
| 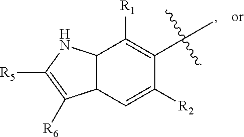 | | |
| 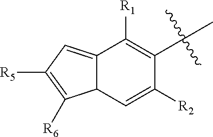 | | |
| 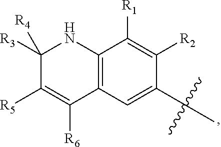 | 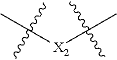 | 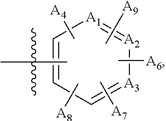 |
| 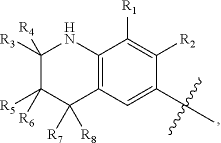 | | 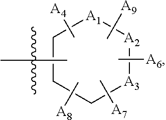 |
| 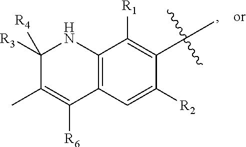 | | 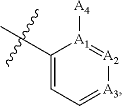 |
| 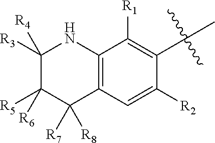 | | 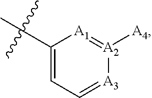 |
| | | 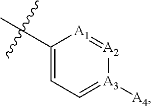 |
| | | 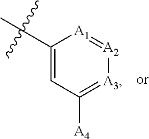 |
| | | 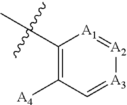 |
| 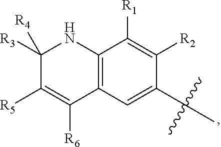 | 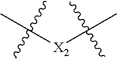 | 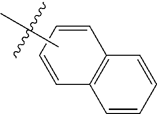 |
| 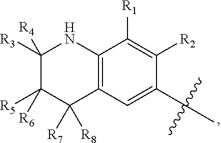 | | |
| 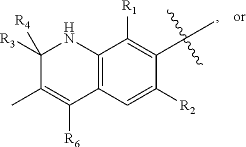 | | |
| 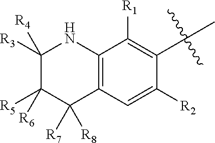 | | |
| 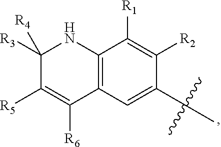 | 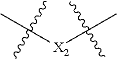 | 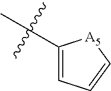 |
| 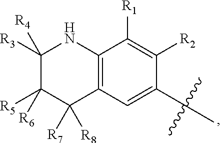 | | |
| 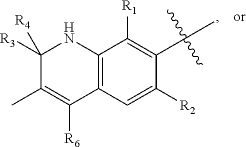 | | |
| 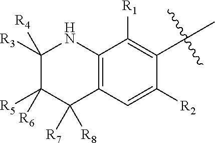 | | |
| 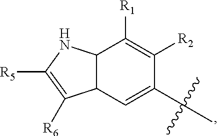 | 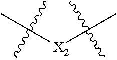 | 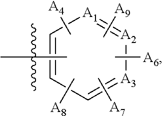 |
| 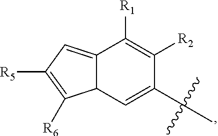 | | 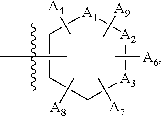 |
| 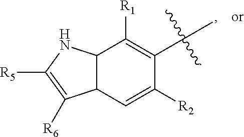 | | 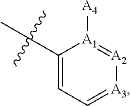 |
| 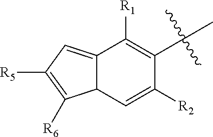 | | 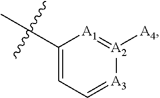 |
| | | 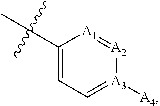 |
| | | 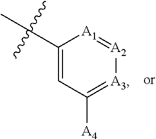 |
| | | 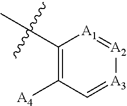 |
| 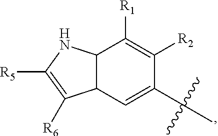 | 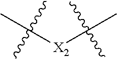 | 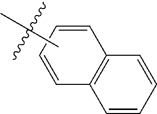 |
| 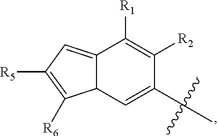 | | |
| 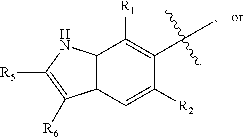 | | |
| 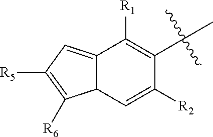 | | |
| 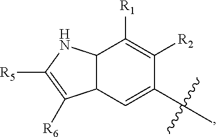 | 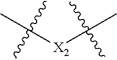 | 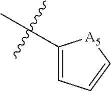 |
| 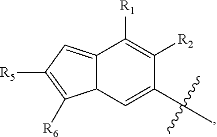 | | |
| 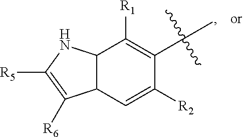 | | |
| 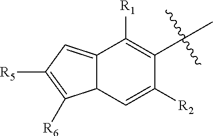 | | |

| | | |
|---|---|---|
| 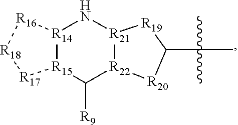 | 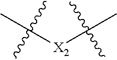 | 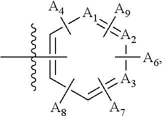 |
| 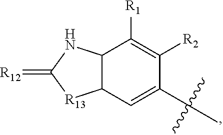 | | 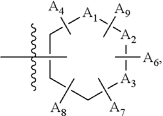 |
| 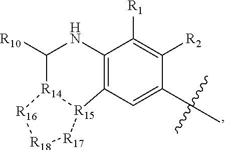 | | 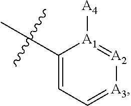 |
| 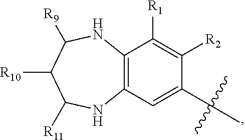 | | 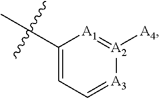 |
| 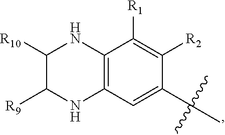 | | 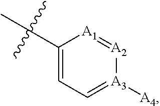 |
| 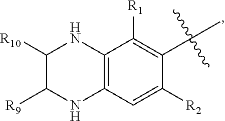 | | 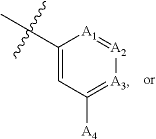 |
| 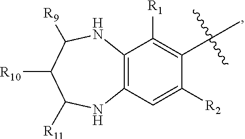 | | 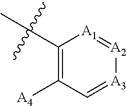 |
| 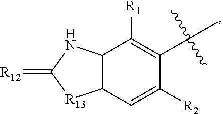 | | |
| 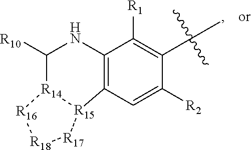 | | |
| 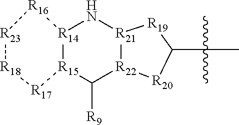 | | |
| 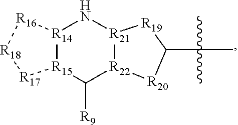 | 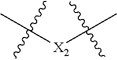 | 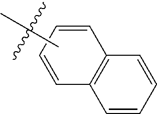 |
| 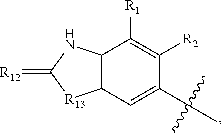 | | |
| 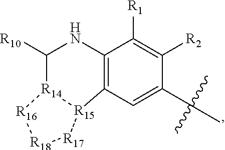 | | |
| 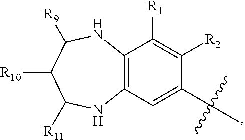 | | |
| 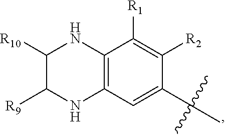 | | |
| 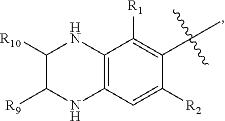 | | |
| 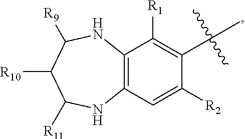 | | |
| 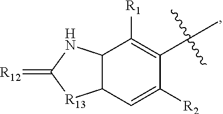 | | |
| 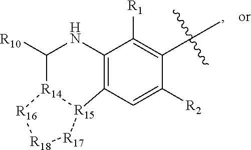 | | |
| 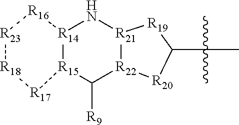 | | |
| 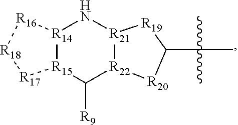 | 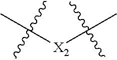 | 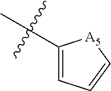 |
| 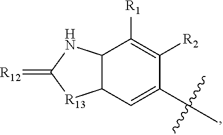 | | |
| 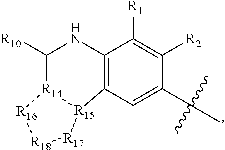 | | |
| 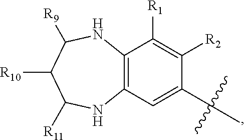 | | |
| 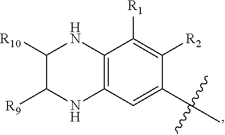 | | |
| 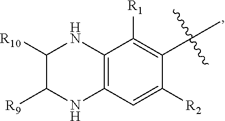 | | |
| 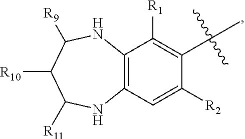 | | |
| 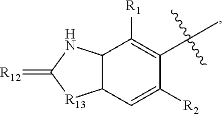 | | |
| 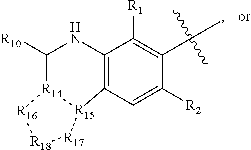 | | |
| 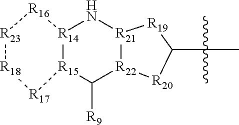 | | |
| 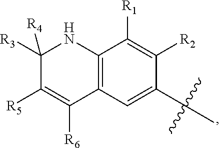 | 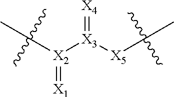 | 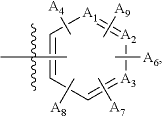 |
| 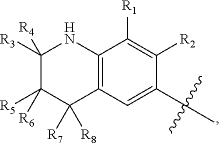 | | 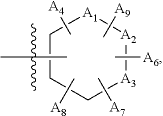 |
| 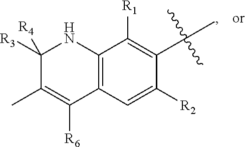 | | 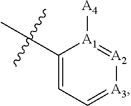 |
| 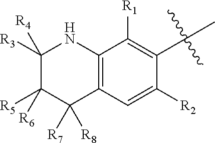 | | 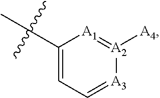 |
| | | 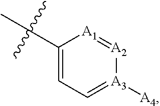 |
| | | 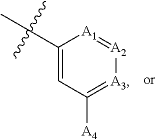 |
| | | 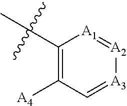 |
| 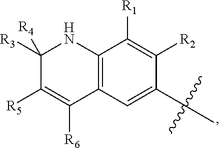 | 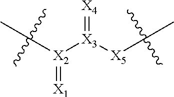 | 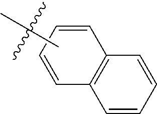 |
| 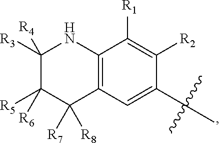 | | |
| 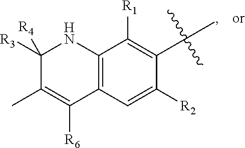 | | |
| 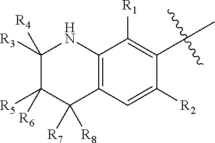 | | |
| 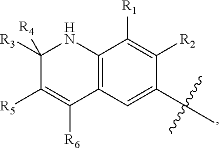 | 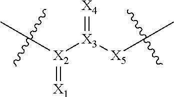 | 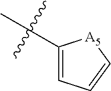 |
| 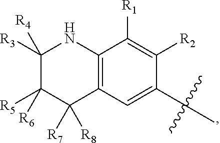 | | |
| 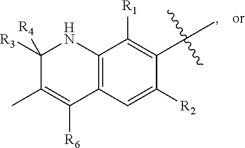 | | |
| 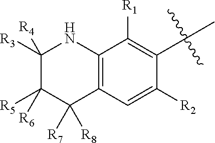 | | |
| 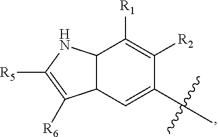 | 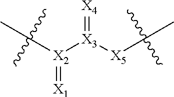 | 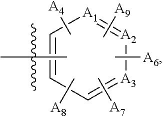 |
| 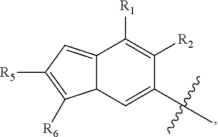 | | 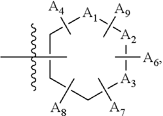 |
| 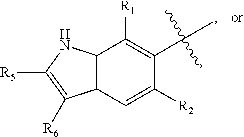 | | 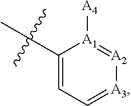 |
| 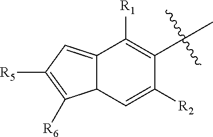 | | 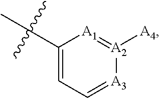 |
| | | 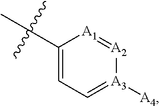 |
| | | 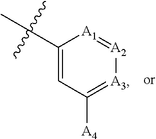 |
| | | 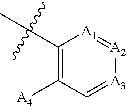 |
| 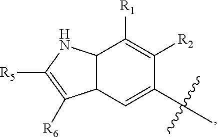 | 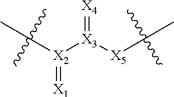 | 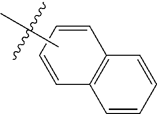 |
| 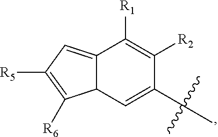 | | |
| 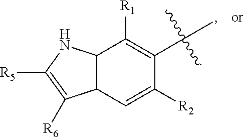 | | |
| 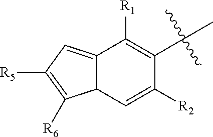 | | |
| 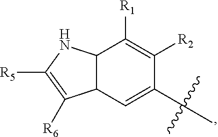 | 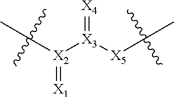 | 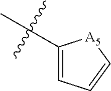 |
| 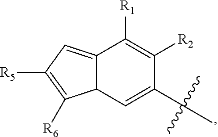 | | |
| 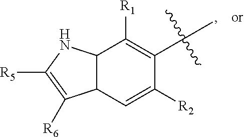 | | |
| 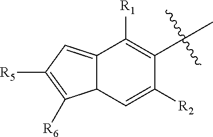 | | |
| 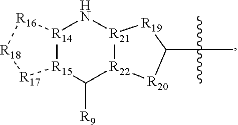 | 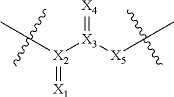 | 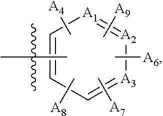 |
| 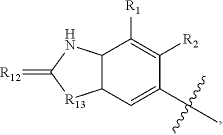 | | 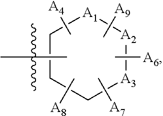 |
| 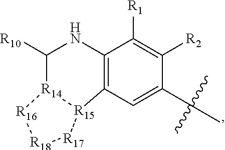 | | 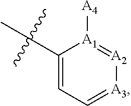 |
| 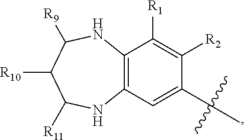 | | 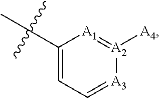 |
| 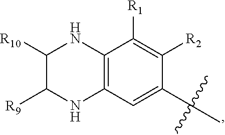 | | 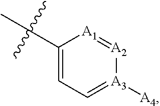 |
| 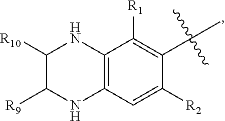 | | 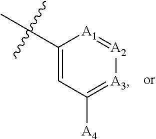 |
| 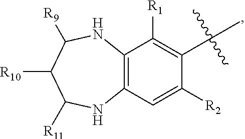 | | 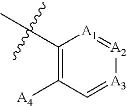 |
| 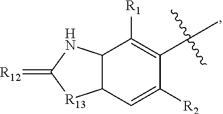 | | |
| 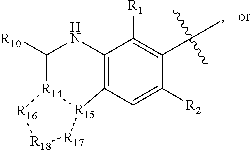 | | |
| 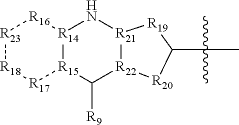 | | |
| 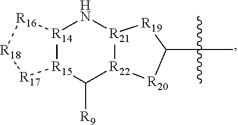 | 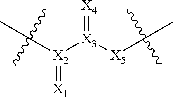 | 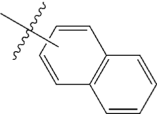 |
| 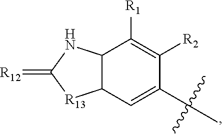 | | |
| 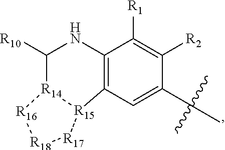 | | |
| 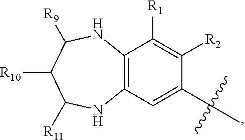 | | |
| 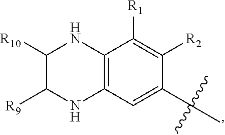 | | |
| 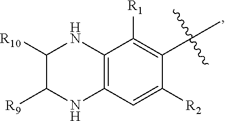 | | |
| 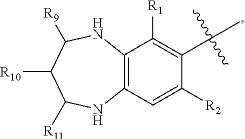 | | |
| 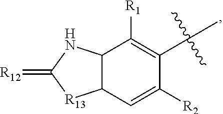 | | |
| 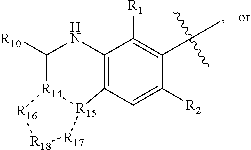 | | |
| 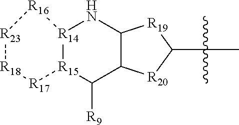 | | |
| 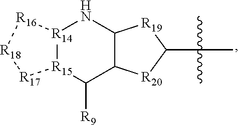 | 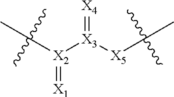 | 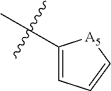 |
| 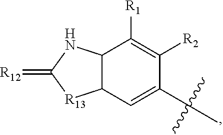 | | |
| 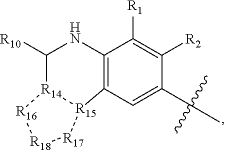 | | |
| 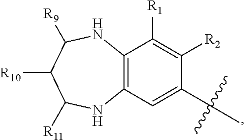 | | |
| 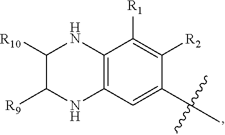 | | |
| 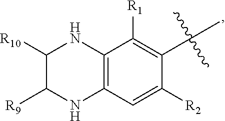 | | |
| 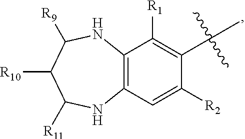 | | |
| 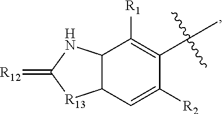 | | |
| 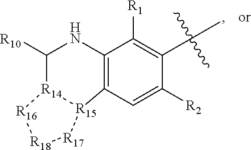 | | |
| 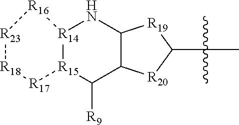 | | |
| 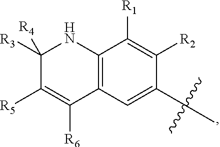 | 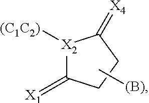 | 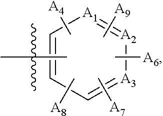 |
| 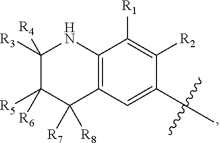 | 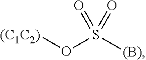 | 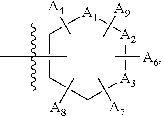 |
| 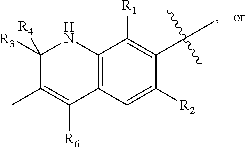 | 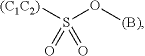 | 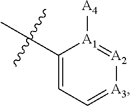 |
| 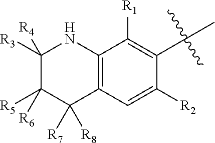 | 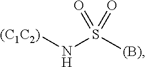 | 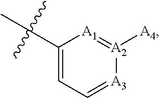 |
| | 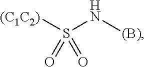 | 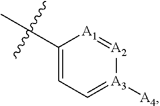 |
| | 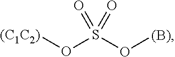 | 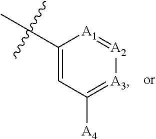 |
| | 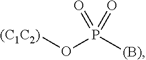 | 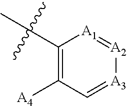 |
| | 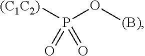 | |
| | 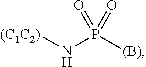 | |
| | 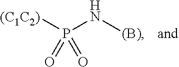 | |
| | 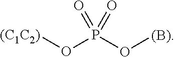 | |
| 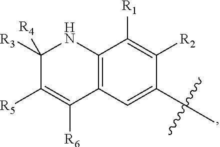 | 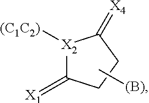 | 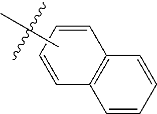 |
| 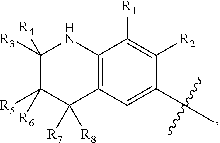 | 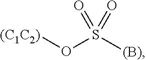 | |
| 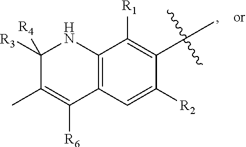 | 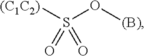 | |
| 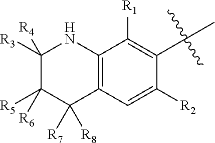 | 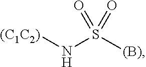 | |
| | 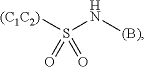 | |
| | 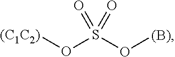 | |
| | 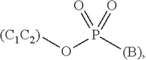 | |
| | 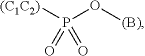 | |
| | 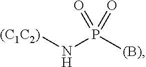 | |
| | 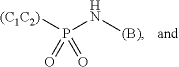 | |
| | 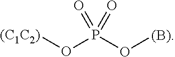 | |
| 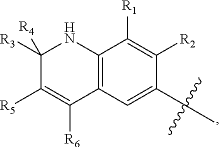 | 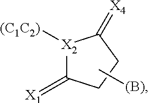 | 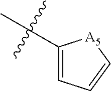 |
| 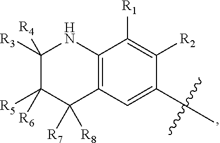 | 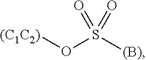 | |
| 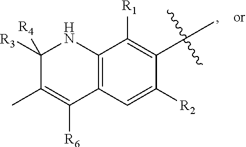 | 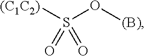 | |
| 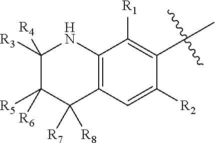 | 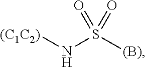 | |
| | 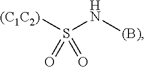 | |
| | 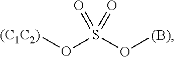 | |
| | 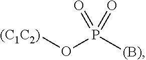 | |
| | 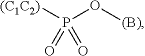 | |
| | 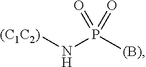 | |
| | 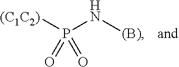 | |
| | 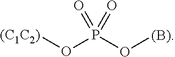 | |
| 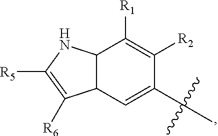 | 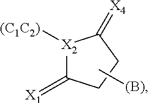 | 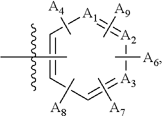 |
| 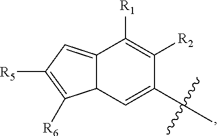 | 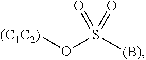 | 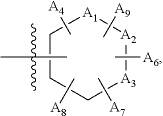 |
| 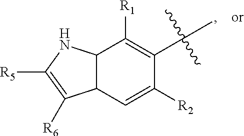 | 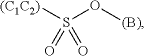 | 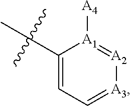 |
| 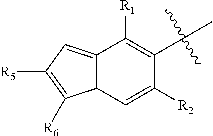 | 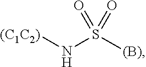 | 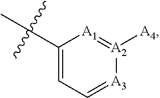 |
| | 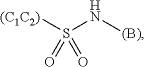 | 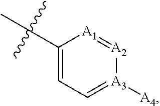 |
| | 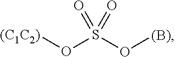 | 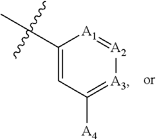 |
| | 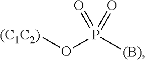 | 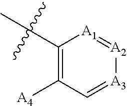 |
| | 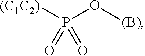 | |
| | 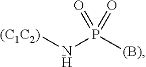 | |
| | 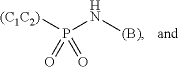 | |
| | 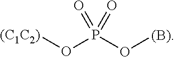 | |
| 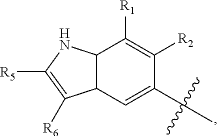 | 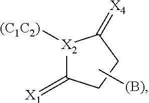 | 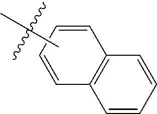 |
| 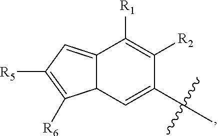 | 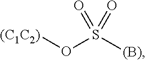 | |
| 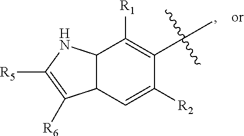 | 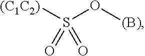 | |
| 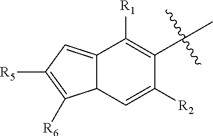 | 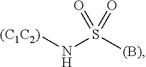 | |
| | 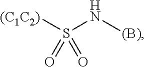 | |
| | 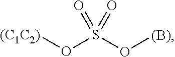 | |
| | 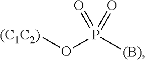 | |
| | 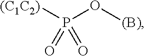 | |
| | 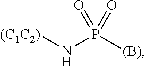 | |
| | 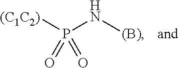 | |
| | 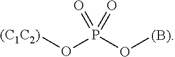 | |
| 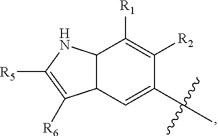 | 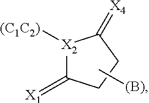 | 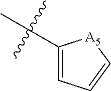 |
| 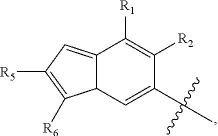 | 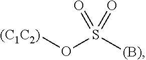 | |
| 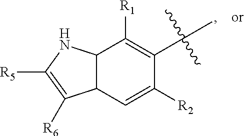 | 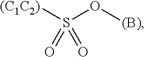 | |
| 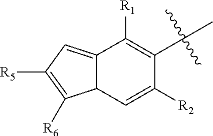 | 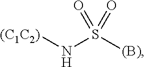 | |
| | 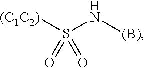 | |
| | 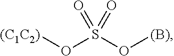 | |
| | 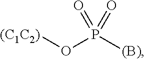 | |
| | 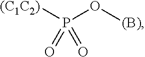 | |
| | 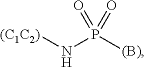 | |
| | 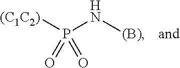 | |
| | 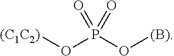 | |
| 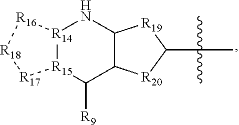 | 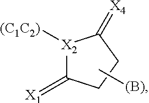 | 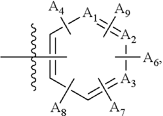 |
| 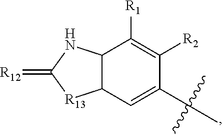 | 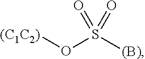 | 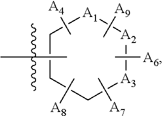 |
| 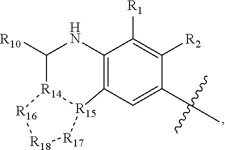 | 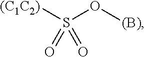 | 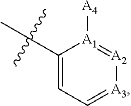 |
| 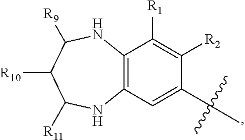 | 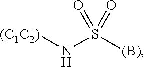 | 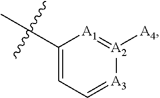 |
| 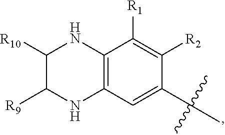 | 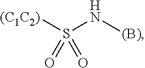 | 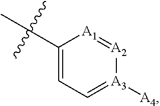 |
| 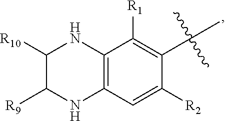 | 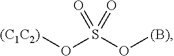 | 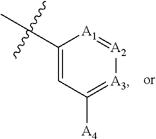 |
| 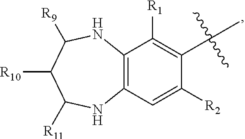 | 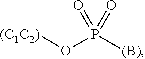 | 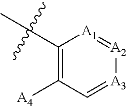 |
| 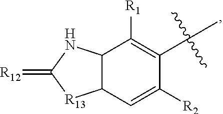 | 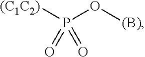 | |
| 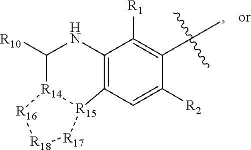 | 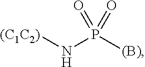 | |
| 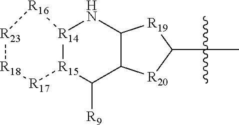 | 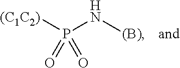 | |

In some embodiments, C1C2-A-B is described by one or more of the following compounds:

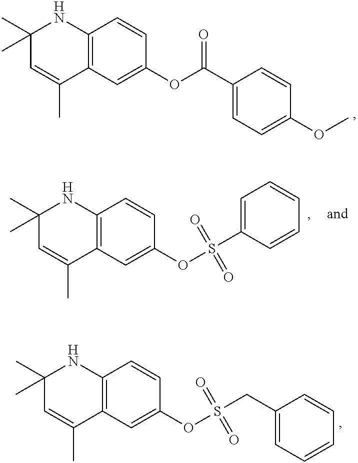

including salts, esters, and prodrugs thereof, and including both R and S enantiomeric forms and racemic mixtures thereof.

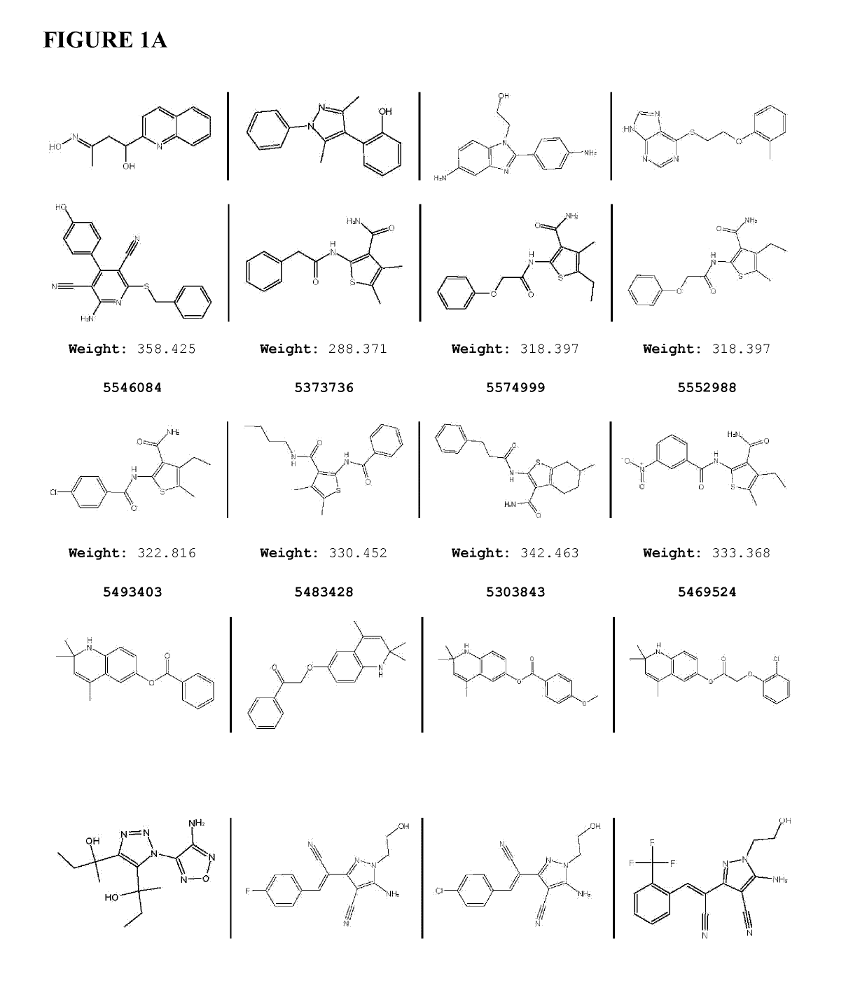

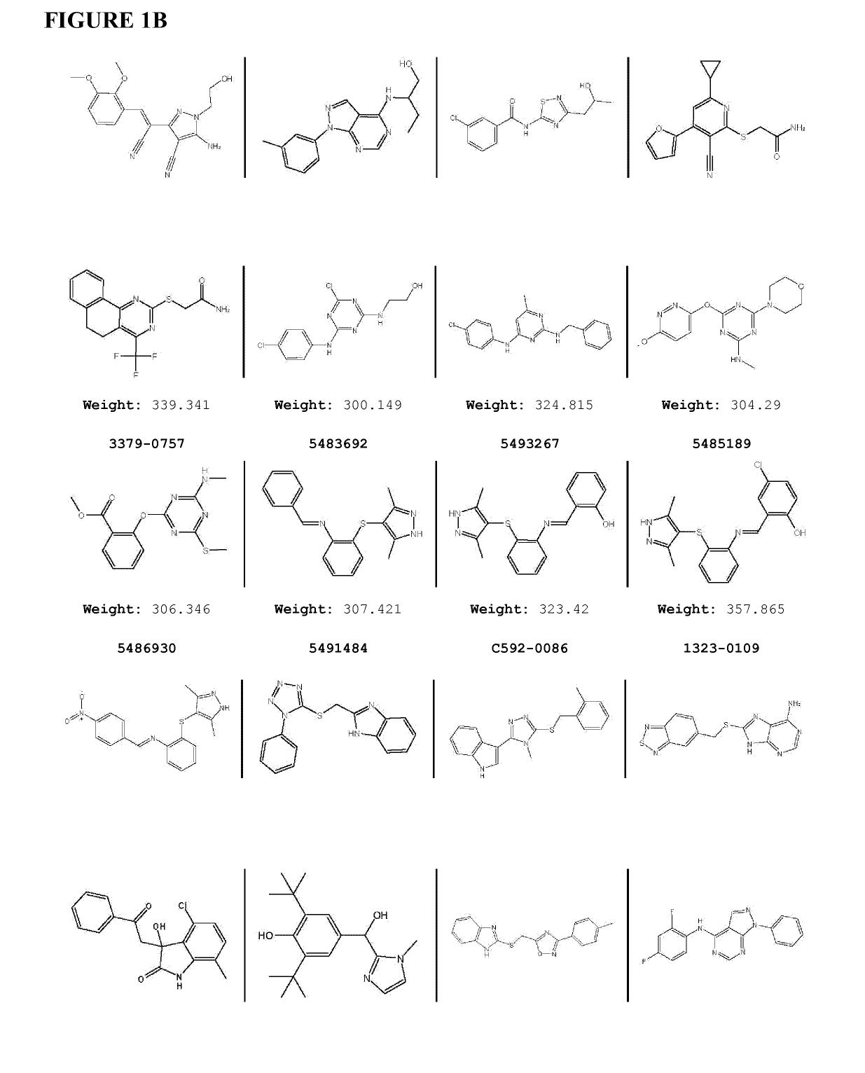

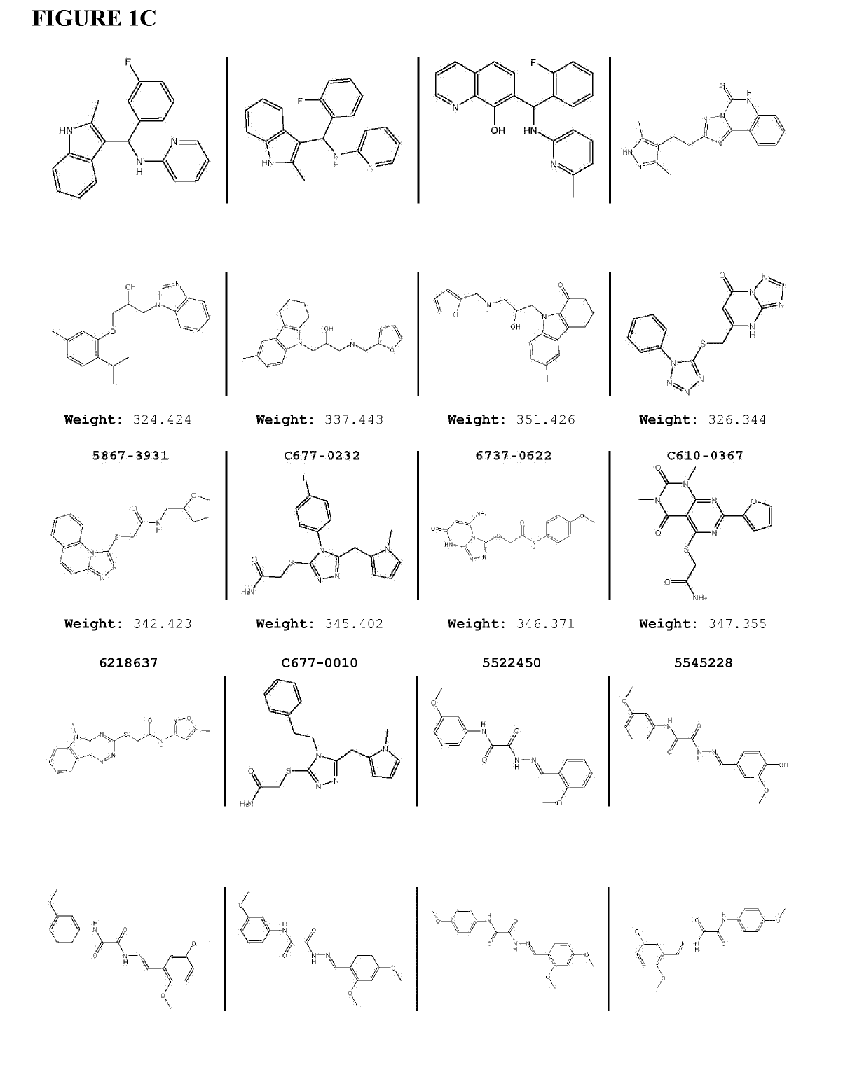

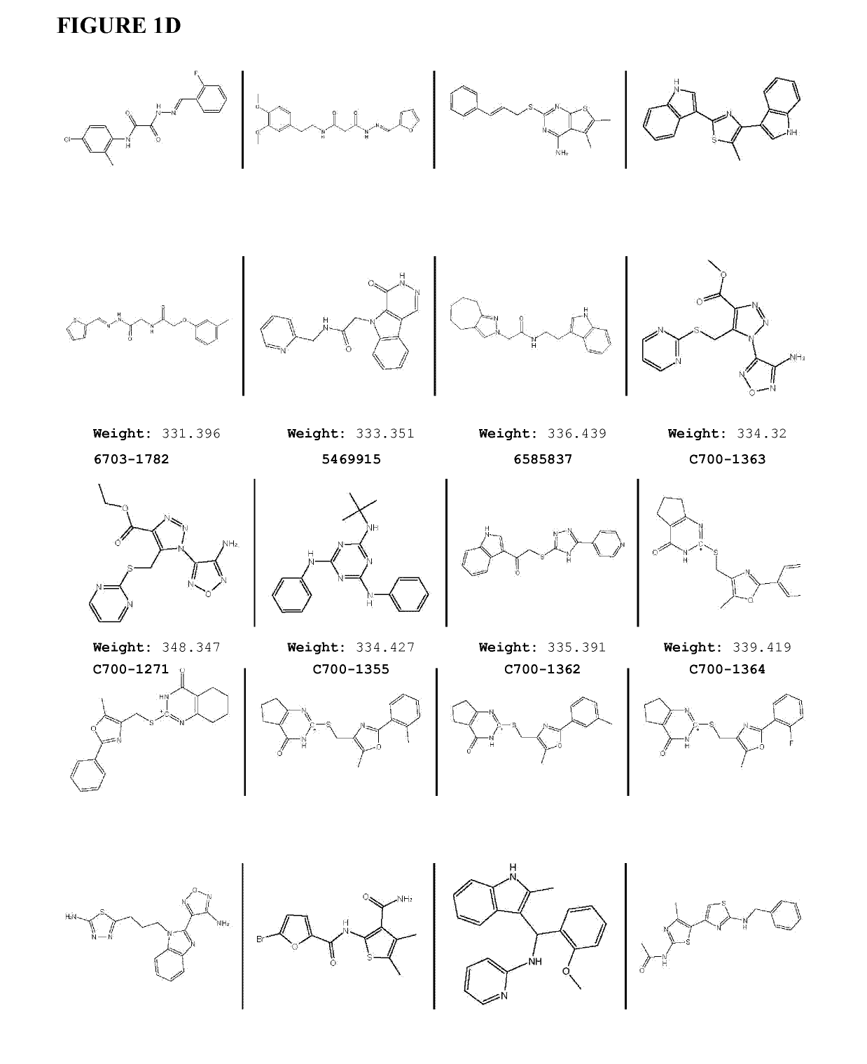

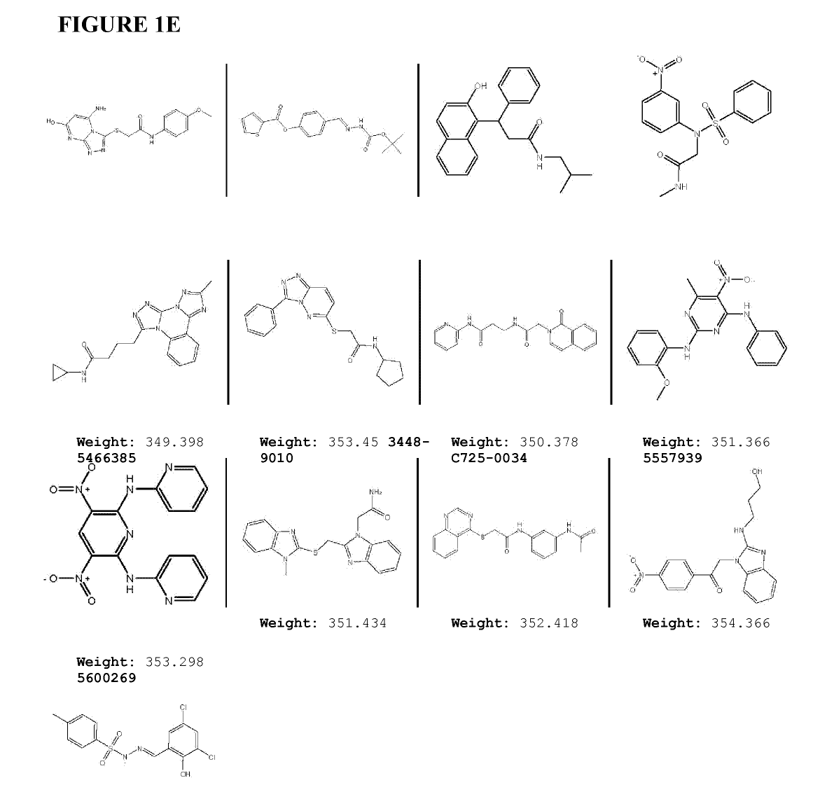

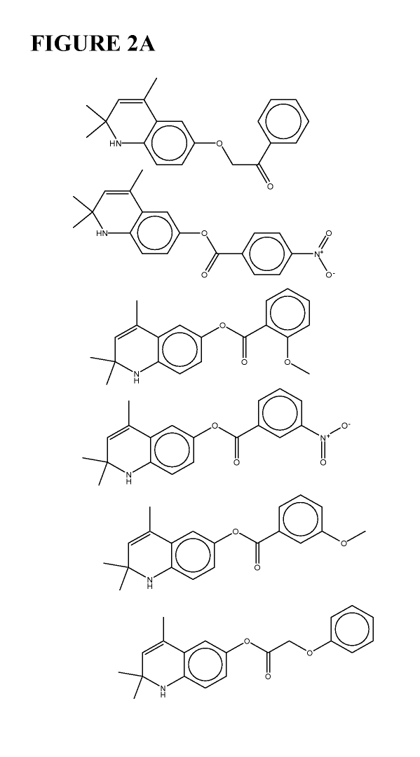

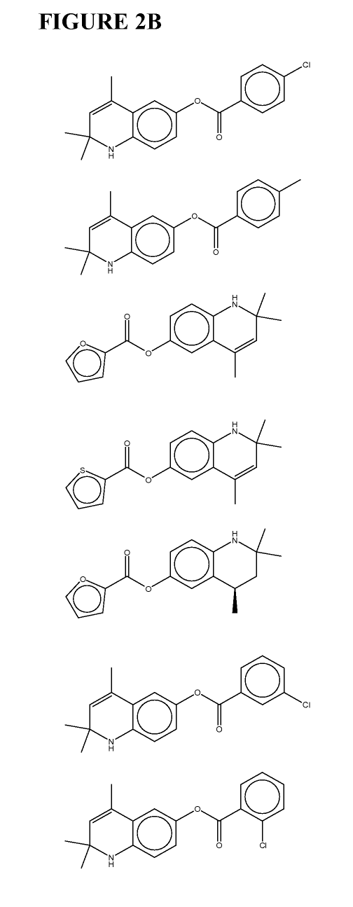

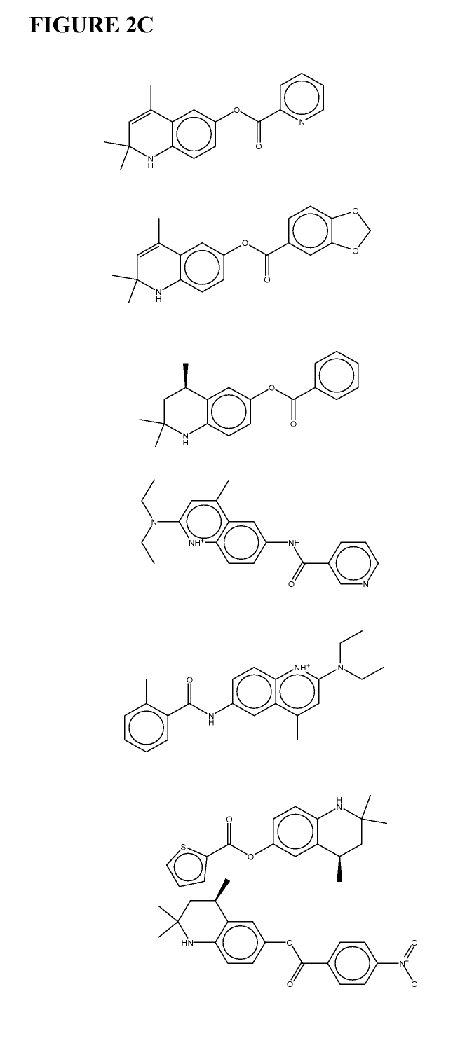

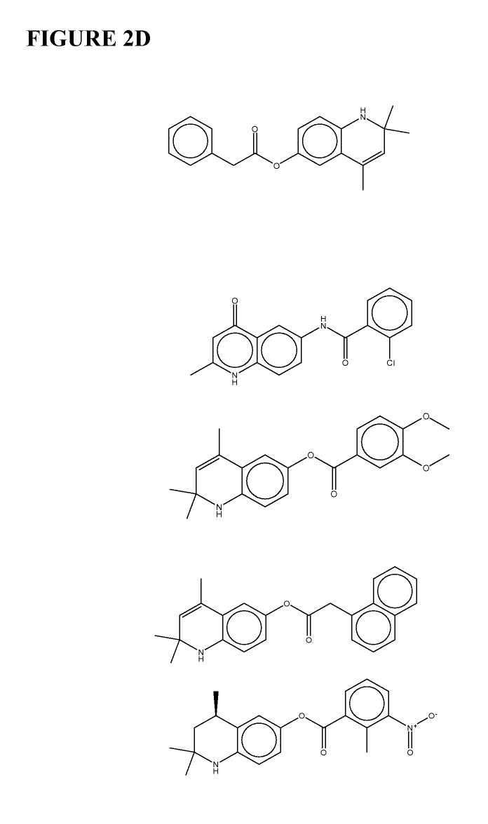

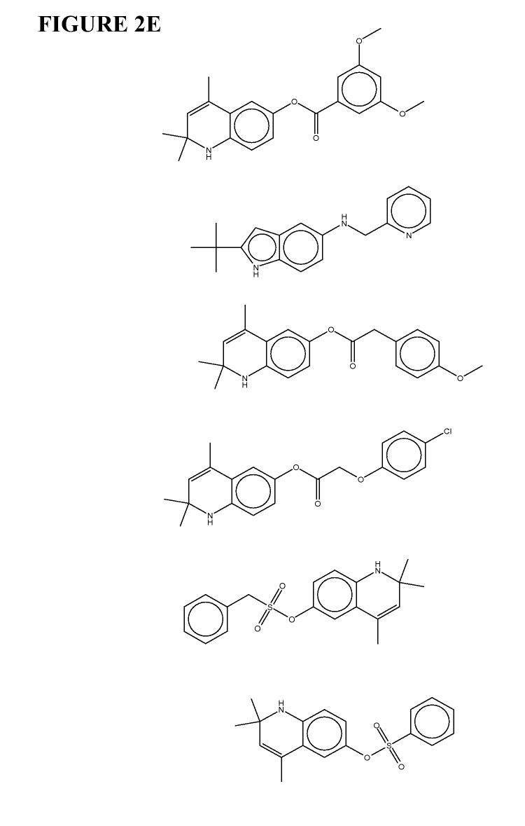

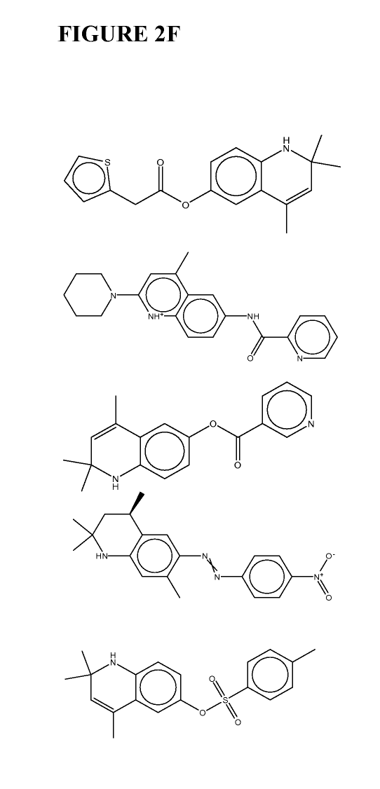

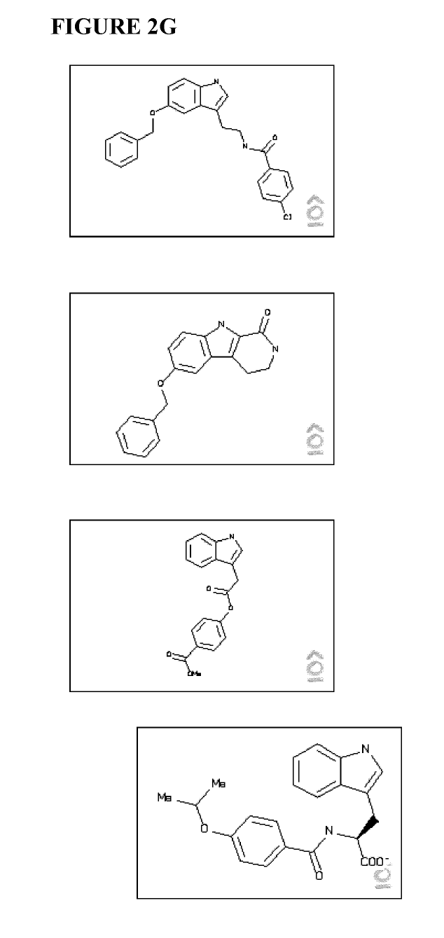

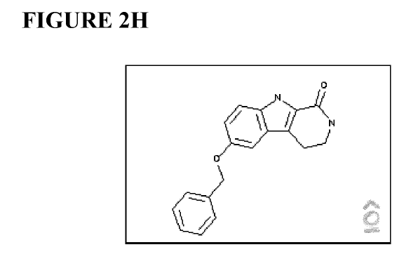

In some embodiments, the compounds are described by one or more of the compounds provided in FIGS. 1, 2 and Table 4.

Any one or more of the compounds can be used to treat a variety of disorders related to HIVp (e.g., AIDS, AIDS Related Complex, HIV Infection, and associated complications and symptoms). Additionally, any one or more of these compounds can be used to inhibit HIVp function through binding the newly identified HIVp binding site. Additionally, any one or more of these compounds can be used in combination with at least one other therapeutic agent known to treat HIV and/or HIV related disorders (e.g., a nucleotide reverse transcriptase inhibitor (e.g., zidovudine (AZT); didanosine (ddI); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)—FTC]; beta-L-FD4 (also called beta-L-D4C and named beta-L-2′,3′-dicleoxy-5-fluoro-cytidene); DAPD, the purine nucleoside, (−)-beta-D-2,6,-diamino-purine dioxolane; and lodenosine (FddA)), a non-nucleoside reverse transcriptase inhibitor (e.g., nevirapine (BI-RG-587); delaviradine (BHAP, U-90152); efavirenz (DMP-266) a benzoxazin-2-one; PNU-142721, a furopyridine-thio-pyrimide; 5-(3,5-dichlorophenyl)-thio-4-isopropyl-1-(4-pyridyl)methyl-I H-imidazol-2-ylmethyl carbonate; MKC442 (1-(ethoxymethyl)-5-(1-methylethyl)-6-(phenylmethyl)-(2,4(1H,3H)-pyrimidi-nedione); and (+)-calanolide A (NSC-675451) and B, coumarin derivatives), a protease inhibitor (e.g., saquinavir (Ro 31-8959); ritonavir (ABT-538); nelfnavir (AG-1343); amprenavir (141W94); lasinavir (BMS-234475); DMP-450; ABT-378; and AG-1549), and an antiviral agent (e.g., hydroxyurea, ribavirin, IL-2, IL-12, pentafuside, and hydroxyurea (Droxia), IL-2, and pentafuside (DP-178, T-20)).

In certain embodiments, the structure of the compounds of the present invention known to bind the newly identified HIVp binding site are chemically connected (e.g., with a biopolymer (e.g., polyethylene glycol or similar agent)) with one or more drugs known to treat a variety of disorders related to HIVp (e.g., AIDS, AIDS Related Complex, HIV Infection, and associated complications and symptoms) (e.g., a nucleotide reverse transcriptase inhibitor, a non-nucleoside reverse transcriptase inhibitor, a protease inhibitor, and/or an antiviral agent). Synthetic polymers, such as polyurethanes, polyesters, polycarbonates, polyureas, polyamides, polyethyleneimines, polyarylene sulfides, polysiloxanes, polyimides, and polyacetates can also form an appropriate agent for chemically connecting the compounds of the present invention known to bind the newly identified HIVp binding site and the one or more drugs known to treat a variety of disorders related to HIVp. In some embodiments, bio-linkers are used to connect the compounds of the present invention known to bind the newly identified HIVp binding site and the one or more drugs known to treat a variety of disorders related to HIVp. Examples of bio-linkers include, but are not limited to, substituted or unsubstituted peptides and polyethers. In some embodiments, such combinations result in a pharmaceutical agent configured to bind HIVp in both the newly identified HIVp binding site and the traditional HIVp binding site.

### III. Pharmaceutical Compositions, Formulations, and Exemplary Administration Routes and Dosing Considerations

Exemplary embodiments of various contemplated medicaments and pharmaceutical compositions are provided below.

A. Preparing Medicaments

It is contemplated that the compounds of the present invention are useful in the preparation of medicaments to treat a variety of conditions associated with HIV and HIV related disorders (e.g., AIDS, AIDS Related Complex, HIV Infection, and associated complications and symptoms).

In addition, it is contemplated that the compounds are also useful for preparing medicaments for treating other disorders wherein the effectiveness of the compounds are known or predicted. The methods and techniques for preparing medicaments of a compound of the present invention are well-known in the art. Exemplary pharmaceutical formulations and routes of delivery are described below.

One of skill in the art will appreciate that any one or more of the compounds described herein, including the many specific embodiments, are prepared by applying standard pharmaceutical manufacturing procedures. Such medicaments can be delivered to the subject by using delivery methods that are well-known in the pharmaceutical arts.

B. Exemplary Pharmaceutical Compositions and Formulation

In some embodiments of the present invention, the compositions are administered alone, while in some other embodiments, the compositions are preferably present in a pharmaceutical formulation comprising at least one active ingredient/agent, as defined above, together with a solid support or alternatively, together with one or more pharmaceutically acceptable carriers and optionally other therapeutic agents. Each carrier must be “acceptable” in the sense that it is compatible with the other ingredients of the formulation and not injurious to the subject.

Contemplated formulations include those suitable oral, rectal, nasal, topical (including transdermal, buccal and sublingual), vaginal, parenteral (including subcutaneous, intramuscular, intravenous and intradermal) and pulmonary administration. In some embodiments, formulations are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present invention suitable for oral administration may be presented as discrete units such as capsules, cachets or tablets, wherein each preferably contains a predetermined amount of the active ingredient; as a powder or granules; as a solution or suspension in an aqueous or non-aqueous liquid; or as an oil-in-water liquid emulsion or a water-in-oil liquid emulsion. In other embodiments, the active ingredient is presented as a bolus, electuary, or paste, etc.

In some embodiments, tablets comprise at least one active ingredient and optionally one or more accessory agents/carriers are made by compressing or molding the respective agents. In some embodiments, compressed tablets are prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose)surface-active or dispersing agent. Molded tablets are made by molding in a suitable machine a mixture of the powdered compound (e.g., active ingredient) moistened with an inert liquid diluent. Tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention are optionally formulated as ointments, creams, suspensions, lotions, powders, solutions, pastes, gels, sprays, aerosols or oils. In alternatively embodiments, topical formulations comprise patches or dressings such as a bandage or adhesive plasters impregnated with active ingredient(s), and optionally one or more excipients or diluents. In some embodiments, the topical formulations include a compound(s) that enhances absorption or penetration of the active agent(s) through the skin or other affected areas. Examples of such dermal penetration enhancers include dimethylsulfoxide (DMSO) and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this invention are constituted from known ingredients in an known manner. This phase typically comprises a lone emulsifier (otherwise known as an emulgent), it is also desirable in some embodiments for this phase to further comprises a mixture of at least one emulsifier with a fat or an oil or with both a fat and an oil.

Preferably, a hydrophilic emulsifier is included together with a lipophilic emulsifier so as to act as a stabilizer. In some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate.

Formulations suitable for vaginal administration may be presented as pessaries, creams, gels, pastes, foams or spray formulations containing in addition to the agent, such carriers as are known in the art to be appropriate.

Formulations suitable for nasal administration, wherein the carrier is a solid, include coarse powders having a particle size, for example, in the range of about 20 to about 500 microns which are administered in the manner in which snuff is taken, i.e., by rapid inhalation (e.g., forced) through the nasal passage from a container of the powder held close up to the nose. Other suitable formulations wherein the carrier is a liquid for administration include, but are not limited to, nasal sprays, drops, or aerosols by nebulizer, an include aqueous or oily solutions of the agents.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. In some embodiments, the formulations are presented/formulated in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of an agent.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, and stabilizers, etc.

In some embodiments, the compounds of the present invention are provided in unsolvated form or are in non-aqueous solutions (e.g., ethanol). The compounds may be generated to allow such formulations through the production of specific crystalline polymorphs compatible with the formulations.

In certain embodiments, the present invention provides instructions for administering said compound to a subject. In certain embodiments, the present invention provides instructions for using the compositions contained in a kit for the treatment of conditions characterized by the dysregulation of apoptotic processes in a cell or tissue (e.g., providing dosing, route of administration, decision trees for treating physicians for correlating patient-specific characteristics with therapeutic courses of action). In certain embodiments, the present invention provides instructions for using the compositions contained in the kit to treat a variety of disorders related to HIVp (e.g., AIDS, AIDS Related Complex, HIV Infection, and associated complications and symptoms).

### C. Exemplary Administration Routes and Dosing Considerations

Various delivery systems are known and can be used to administer therapeutic agents (e.g., exemplary compounds as described in Section II above) of the present invention, e.g., encapsulation in liposomes, microparticles, microcapsules, receptor-mediated endocytosis, and the like. Methods of delivery include, but are not limited to, intra-arterial, intra-muscular, intravenous, intranasal, and oral routes. In specific embodiments, it may be desirable to administer the pharmaceutical compositions of the invention locally to the area in need of treatment; this may be achieved by, for example, and not by way of limitation, local infusion during surgery, injection, or by means of a catheter.

It is contemplated that the agents identified can be administered to subjects or individuals susceptible to or at risk of developing a variety of disorders related to HIVp (e.g., AIDS, AIDS Related Complex, HIV Infection, and associated complications and symptoms). When the agent is administered to a subject such as a mouse, a rat or a human patient, the agent can be added to a pharmaceutically acceptable carrier and systemically or topically administered to the subject. To determine patients that can be beneficially treated, a tissue sample is removed from the patient and the cells are assayed for sensitivity to the agent.

Therapeutic amounts are empirically determined and vary with the pathology being treated, the subject being treated and the efficacy and toxicity of the agent. When delivered to an animal, the method is useful to further confirm efficacy of the agent.

In some embodiments, in vivo administration is effected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

Suitable dosage formulations and methods of administering the agents are readily determined by those of skill in the art. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., a protease inhibitor), the effective amount may be more or less than when the agent is used alone.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including, but not limited to, oral, rectal, nasal, topical (including, but not limited to, transdermal, aerosol, buccal and sublingual), vaginal, parental (including, but not limited to, subcutaneous, intramuscular, intravenous and intradermal) and pulmonary. It is also appreciated that the preferred route varies with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient.

Desirable blood levels of the agent may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

D. Exemplary Co-Administration Routes and Dosing Considerations

The present invention also includes methods involving co-administration of the compounds described herein with one or more additional active agents. Indeed, it is a further aspect of this invention to provide methods for enhancing prior art therapies and/or pharmaceutical compositions by co-administering a compound of this invention. In co-administration procedures, the agents may be administered concurrently or sequentially. In one embodiment, the compounds described herein are administered prior to the other active agent(s). The pharmaceutical formulations and modes of administration may be any of those described above. In addition, the two or more co-administered chemical agents, biological agents or radiation may each be administered using different modes or different formulations.

The agent or agents to be co-administered depends on the type of condition being treated. For example, when the condition being treated is HIV and/or an HIV related disorder (e.g., AIDS, AIDS Related Complex, HIV Infection, and associated complications and symptoms) the agent is known to treat HIV and/or HIV related disorders (e.g., a nucleotide reverse transcriptase inhibitor (e.g., zidovudine (AZT); didanosine (ddI); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)—FTC]; beta-L-FD4 (also called beta-L-D4C and named beta-L-2′,3′-dicleoxy-5-fluoro-cytidene); DAPD, the purine nucleoside, (−)-beta-D-2,6,-diamino-purine dioxolane; and lodenosine (FddA)), a non-nucleoside reverse transcriptase inhibitor (e.g., nevirapine (BI-RG-587); delaviradine (BHAP, U-90152); efavirenz (DMP-266) a benzoxazin-2-one; PNU-142721, a furopyridine-thio-pyrimide; 5-(3,5-dichlorophenyl)-thio-4-isopropyl-1-(4-pyridyl)methyl-I H-imidazol-2-ylmethyl carbonate; MKC442 (1-(ethoxymethyl)-5-(1-methylethyl)-6-(phenylmethyl)-(2,4(1H,3H)-pyrimidi-nedione); and (+)-calanolide A (NSC-675451) and B, coumarin derivatives), a protease inhibitor (e.g., saquinavir (Ro 31-8959); ritonavir (ABT-538); nelfnavir (AG-1343); amprenavir (141W94); lasinavir (BMS-234475); DMP-450; ABT-378; and AG-1549), and an antiviral agent (e.g., hydroxyurea, ribavirin, IL-2, IL-12, pentafuside, and hydroxyurea (Droxia), IL-2, and pentafuside (DP-178, T-20)). The additional agents to be co-administered can be any of the well-known agents in the art, including, but not limited to, those that are currently in clinical use. The determination of appropriate type and dosage of radiation treatment is also within the skill in the art or can be determined with relative ease.

In some embodiments, the additional agent(s) is a “nucleoside and nucleotide reverse transcriptase inhibitor” (“NRTI”) (e.g., zidovudine (AZT); didanosine (ddI); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)—FTC]; beta-L-FD4 (also called beta-L-D4C and named beta-L-2′,3′-dicleoxy-5-fluoro-cytidene); DAPD, the purine nucleoside, (−)-beta-D-2,6,-diamino-purine dioxolane; and lodenosine (FddA)).

In some embodiments, the additional agent(s) is a “non-nucleoside reverse transcriptase inhibitor” (“NNRTI”) (e.g., nevirapine (BI-RG-587); delaviradine (BHAP, U-90152); efavirenz (DMP-266) a benzoxazin-2-one; PNU-142721, a furopyridine-thio-pyrimide; 5-(3,5-dichlorophenyl)-thio-4-isopropyl-1-(4-pyridyl)methyl-I H-imidazol-2-ylmethyl carbonate; MKC442 (1-(ethoxymethyl)-5-(1-methylethyl)-6-(phenylmethyl)-(2,4(1H,3H)-pyrimidi-nedione); and (+)-calanolide A (NSC-675451) and B, coumarin derivatives).

In some embodiments, the additional agent(s) is a “protease inhibitor” (“PI”) (e.g., saquinavir (Ro 31-8959); ritonavir (ABT-538); nelfnavir (AG-1343); amprenavir (141W94); lasinavir (BMS-234475); DMP-450; ABT-378; and AG-1549).

In some embodiments, the additional agent(s) is an other antiviral agent including, but not limited to, hydroxyurea, ribavirin, IL-2, IL-12, pentafuside, and hydroxyurea (Droxia), IL-2, and pentafuside (DP-178, T-20).

### IV. Drug Screens

In some embodiments of the present invention, the compounds of the present invention, and other potentially useful compounds, are screened for their binding affinity to the newly identified HIVp binding site. In particularly preferred embodiments, compounds are selected for use in the methods of the present invention by measuring their biding affinity to the newly identified HIVp binding site. A number of suitable screens for measuring the binding affinity of drugs and other small molecules to enzymes such as HIVp are known in the art. In some embodiments, binding affinity screens are conducted in in vitro systems. In other embodiments, these screens are conducted in in vivo or ex vivo systems. In some embodiments, compounds of the present invention, and other potentially useful compounds, are screened for an ability to displace compounds bound in the newly identified HIVp binding site.

In some embodiments, compounds of the present invention, and other potentially useful compounds, are screened for an ability to inhibit the function of HIVp (e g., inhibiting HIVp enzyme activity, inhibiting maturation of HIV particles, terminating replication of the HIV virus) through binding the newly identified HIVp binding site.

In some embodiments, compounds of the present invention, and other potentially useful compounds, are screened for an ability to treat disorders associated with HIVp (e.g., AIDS, AIDS Related Complex, HIV Infection, and associated complications and symptoms) through binding the newly identified HIVp binding site.

Additional embodiments are directed to measuring levels (e.g., intracellular) of HIVp activity (e.g., HIVp enzyme function) in cells and/or tissues to measure the effectiveness of particular contemplated methods and compounds of the present invention. In this regard, those skilled in the art will appreciate and be able to provide a number of assays and methods useful for measuring HIVp activity in cells and/or tissues.

In some embodiments, structure-based virtual screening methodologies are contemplated for predicting the binding affinity of compounds of the present invention with the newly identified HIVp binding site. In some embodiments, compound structures are predicted from a molecular modeling software (e.g., MacroModel, MOE, Glide, Gold, Autodock, DOCK, Unity, Cerius2, Daylight, PipelinePilot, ChemAxon, Sprout, Hook, MCSS, AMBER, BOSS).

Any suitable assay that allows for a measurement of the HIVp enzyme function or the affinity of an exemplary compound of the present invention to the newly identified HIVp binding site may be utilized. Examples include, but are not limited to, competition binding using an exemplary compound, surface plasma resonance (SPR) and radio-immunopreciptiation assays (Lowman et al., J. Biol. Chem. 266:10982 [1991]). Surface Plasmon Resonance techniques involve a surface coated with a thin film of a conductive metal, such as gold, silver, chrome or aluminum, in which electromagnetic waves, called Surface Plasmons, can be induced by a beam of light incident on the metal glass interface at a specific angle called the Surface Plasmon Resonance angle. Modulation of the refractive index of the interfacial region between the solution and the metal surface following binding of the captured macromolecules causes a change in the SPR angle which can either be measured directly or which causes the amount of light reflected from the underside of the metal surface to change. Such changes can be directly related to the mass and other optical properties of the molecules binding to the SPR device surface. Several biosensor systems based on such principles have been disclosed (See e.g., WO 90/05305). There are also several commercially available SPR biosensors (e.g., BiaCore, Uppsala, Sweden).

In some embodiments, detection of compounds able to bind the newly identified HIVp binding site involves detecting variations in the melting temperature of HIVp in the presence and absence of such compounds (see, e.g., U.S. Pat. No. 6,303,322; herein incorporated by reference in its entirety). For example, in some embodiments, a variation in the melting temperature of HIVp in the presence of a compound indicates binding of such a compound with HIVp.

The present invention also provides methods of modifying and derivatizing the compositions of the present invention to increase desirable properties (e.g., ability to inhibit HIVp enzyme activity, ability to inhibit maturation of HIV particles) or to minimize undesirable properties. The principles of chemical derivatization are well understood. In some embodiments, iterative design and chemical synthesis approaches are used to produce a library of derivatized child compounds from a parent compound.

### V. Therapeutic Application

In certain embodiments, the present invention provides methods (e.g., therapeutic applications) for treating disorders related to HIVp (e.g., AIDS, AIDS Related Complex, HIV Infection, and associated complications and symptoms). In some embodiments, the methods involve administering one or more compound (see, e.g., Section II-Exemplary Compounds) of the present invention to a subject having an HIVp related disorder wherein the compounds target the newly identified HIVp binding site. In such embodiments, binding of the newly identified HIVp binding site modulates (e.g., inhibits) the function of HIVp (e.g., HIVp enzyme activity, ability of HIVp to promote maturation of HIV particles). In some embodiments, modulation (e g., inhibition) of HIVp results is associated with a general reduction of symptoms associated with disorders related to HIVp (e.g., improved immune function). In some embodiments, modulation (e g., inhibition) of HIVp results in elimination of symptoms associated with HIVp.

### VI. Detection of HIV Protease Related Disorders

The present invention provides methods of detecting (e.g., diagnosing) the presence or absence of a HIV protease related disorder disorders associated with HIV protease (e.g., AIDS, AIDS Related Complex, and HIV Infection). The present invention is not limited to a particular method or technique for detecting the presence or absence of a HIV protease related disorder. In some embodiments, one or more of the compounds of the present invention are used as probes within a sample (e.g., blood sample, urine sample) (e.g., a sample from a subject) to detect the presence of HIV protease within the sample. In some embodiments, such knowledge would be beneficial in terms of diagnosing exposure of a subject to HIV, and/or in diagnosing HIV and/or AIDS.

### Experimental

### EXAMPLE 1

The following compounds shown in Table 4 were found to inhibit HIV-1 protease by 25% or more at 150 μM (i.e., showed ≦75% activity in the assay).

TABLE 4

| | Remain | | |
|---|---|---|---|
| Compound | Activity | Stdev | Weight |
| 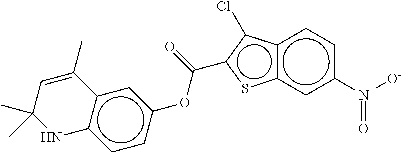 | 56.93 | 5.82 | 428.9 |
| 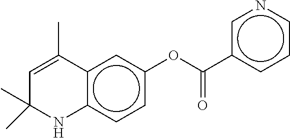 | 58.86 | 8.63 | 294.4 |
| 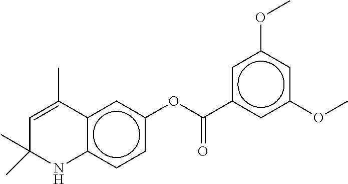 | 59.22 | 4.37 | 353.4 |
| 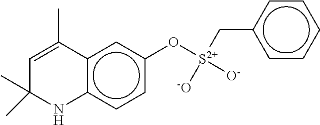 | 59.59 | 42.59 | 343.4 |
| 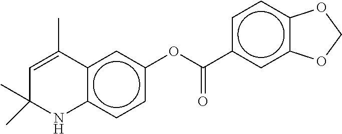 | 63.58 | 12.52 | 337.4 |
| 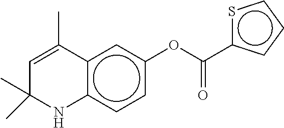 | 64.08 | 4.77 | 299.4 |
| 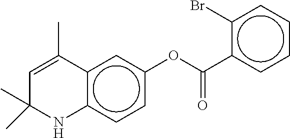 | 65.33 | 9 | 372.3 |
| 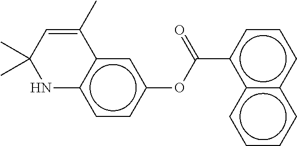 | 65.34 | 6.75 | 343.4 |
| 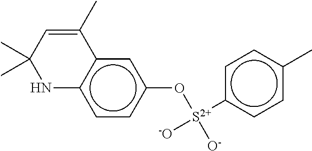 | 67.71 | 12.41 | 343.4 |
| 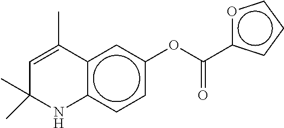 | 69.67 | 5.49 | 283.3 |
| 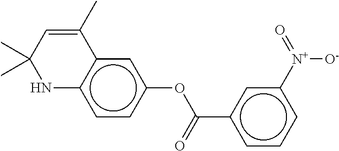 | 73.01 | 7.98 | 338.4 |
| 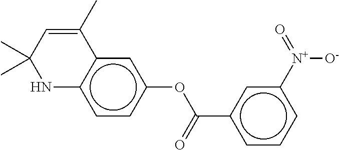 | 73.01 | 7.98 | 338.4 |
| 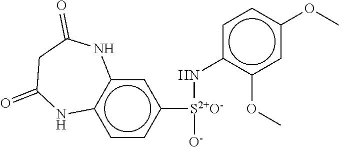 | 62.06 | 5.65 | 390.4 |
| 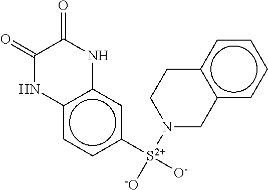 | 68.98 | 10.18 | 357.4 |
| 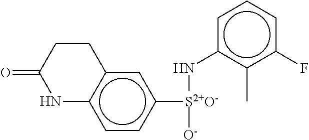 | 71.96 | 8.84 | 333.4 |
| 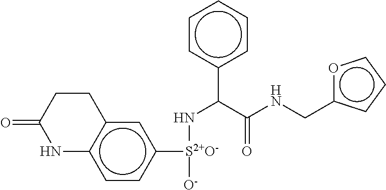 | 65.22 | 7.8 | 439.5 |
| 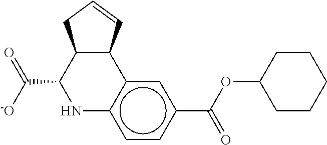 | 42.35 | 3.84 | 340.4 |
| 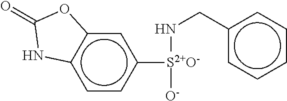 | 74.48 | 6.32 | 304.3 |
| 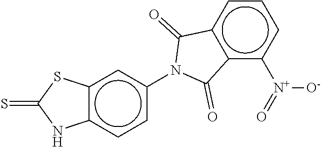 | 36.93 | 5.61 | 357.4 |
| 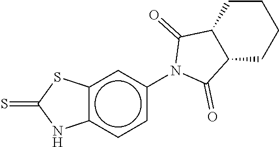 | 51.9 | 8.56 | 318.4 |
| 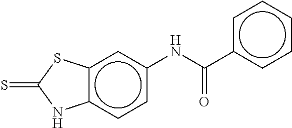 | 65.14 | 2.07 | 286.4 |
| 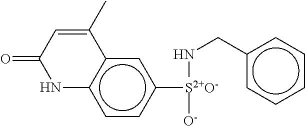 | 67.74 | 6.32 | 328.4 |
| 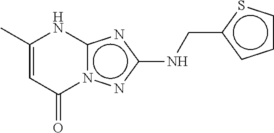 | 63.59 | 3.86 | 261.3 |
| 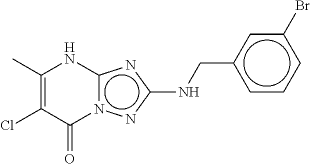 | 71.2 | 7.07 | 368.6 |
| 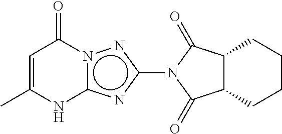 | 67.49 | 6.32 | 301.3 |
| 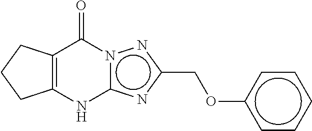 | 57.77 | 5.27 | 282.3 |
| 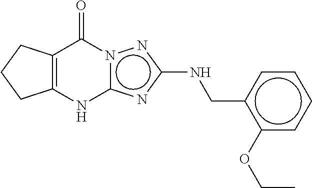 | 75.44 | 3.68 | 325.4 |
| 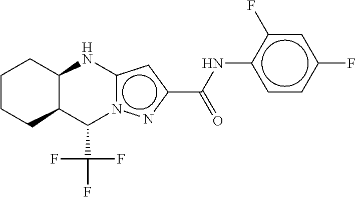 | 64.91 | 6.32 | 400.4 |
| 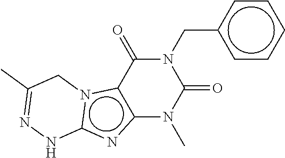 | 65.08 | 7.41 | 324.3 |

All publications and patents mentioned in the above specification are herein incorporated by reference for all purposes. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising one or more compounds that bind a region of HIV protease defined by the following residues: I84, V32, P81, T80, P79, G78, V56, I54, I47, G48, G49, and I50.

2. The composition of claim 1, wherein said compound is selected from the group consisting of: 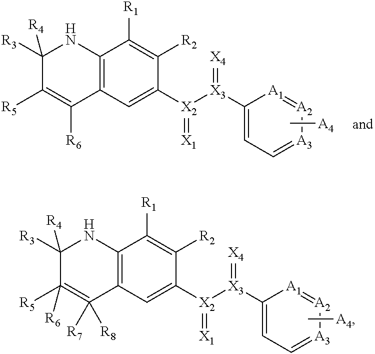
including salts, esters, and prodrugs thereof, and including both R and S enantiomeric forms and racemic mixtures thereof,
wherein R1 and R2 are independently selected from the group consisting of H and OH,
wherein R3, R4, R5, R6, R7, and R8 are independently selected from the group consisting of methyl, ethyl, iodo, bromo, and chloro, or R5, R6, R7, and R8 can include a small hydrophilic group such as hydroxyl,
wherein X1 and X4 are present or absent and, if present, are independently selected from group consisting of O and S,
wherein X2, and X3 independently selected from the group consisting of O, C, S, SO₂, PO₂, or NH,
wherein A1, A2, and A3 are independently selected group consisting of C and N, and
wherein A4 is present or absent, and if present, is selected from the group consisting of:

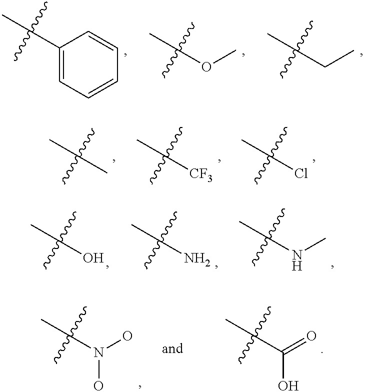

3. The composition of claim 1, further comprising one or more therapeutic agents known to treat disorders associated with HIV protease.

4. The composition of claim 3, wherein said one or more therapeutic agents known to treat disorders associated with HIV protease is selected from the group consisting of a nucleotide reverse transcriptase inhibitor, a non-nucleoside reverse transcriptase inhibitor, a protease inhibitor, and an antiviral agent.

5. The composition of claim 3, wherein said one or more therapeutic agents known to treat disorders associated with HIV protease is selected from the group consisting of zidovudine (AZT); didanosine (ddI); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)—FTC]; beta-L-FD4 (also called beta-L-D4C and named beta-L-2′,3′-dicleoxy-5-fluoro-cytidene); DAPD, the purine nucleoside, (−)-beta-D-2,6,-diamino-purine dioxolane; and lodenosine (FddA)), nevirapine (BI-RG-587); delaviradine (BHAP, U-90152); efavirenz (DMP-266) a benzoxazin-2-one; PNU-142721, a furopyridine-thio-pyrimide; 5-(3,5-dichlorophenyl)-thio-4-isopropyl-1-(4-pyridyl)methyl-I H-imidazol-2-ylmethyl carbonate; MKC442 (1-(ethoxymethyl)-5-(1-methylethyl)-6-(phenylmethyl)-(2,4(1H,3H)-pyrimidi-nedione); and (+)-calanolide A (NSC-675451) and B, coumarin derivatives), saquinavir (Ro 31-8959); ritonavir (ABT-538); nelihavir (AG-1343); amprenavir (141W94); lasinavir (BMS-234475); DMP-450; ABT-378; and AG-1549), hydroxyurea, ribavirin, IL-2, IL-12, pentafuside, and hydroxyurea (Droxia), IL-2, and pentafuside (DP-178, T-20).

6. The composition of claim 1, wherein said compound is described by the formula: C1C2-A-B, including salts, esters, and prodrugs thereof, and including both R and S enantiomeric forms and racemic mixtures thereof, wherein said compound comprises at least six of the following seven characteristics: three aromatic groups, one at position C1, one at position C2, one at position B, two hydrophobic groups, one hydrogen bond donor group, and one hydrogen bond acceptor group, wherein A provides a covalent linker connecting C1C2 and B.

7. The composition of claim 6, wherein C1C2 is selected from the group consisting of:

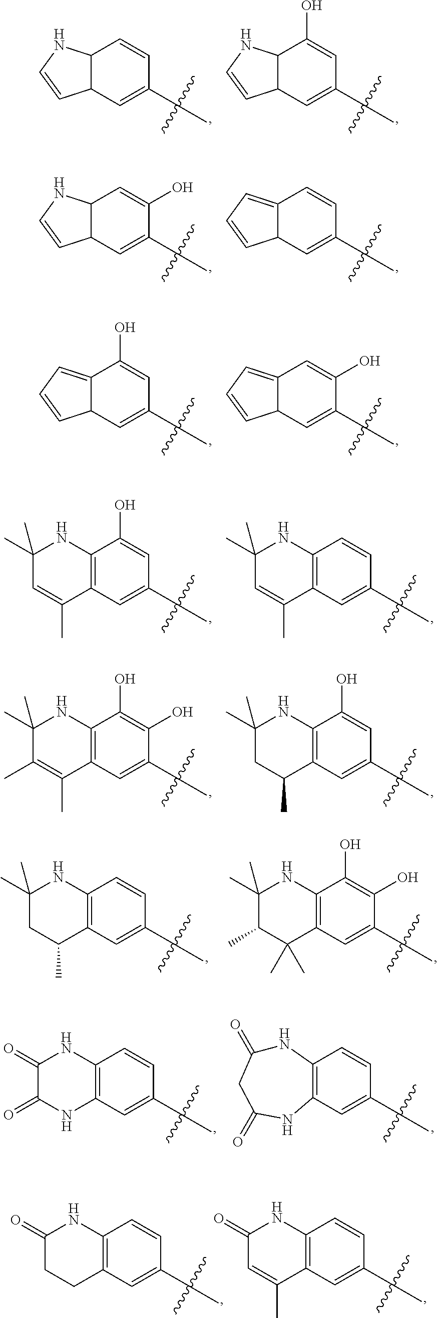

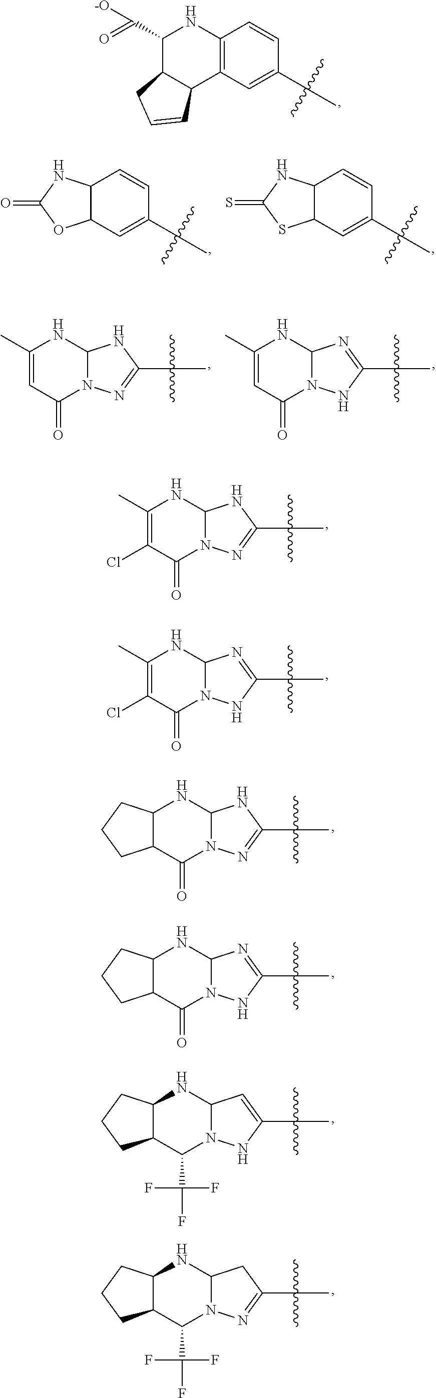

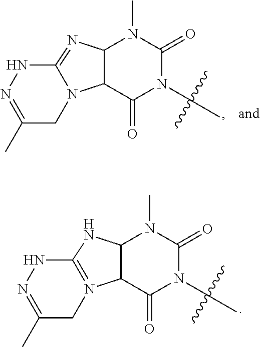

8. The composition of claim 6, wherein B is selected from the group consisting of:

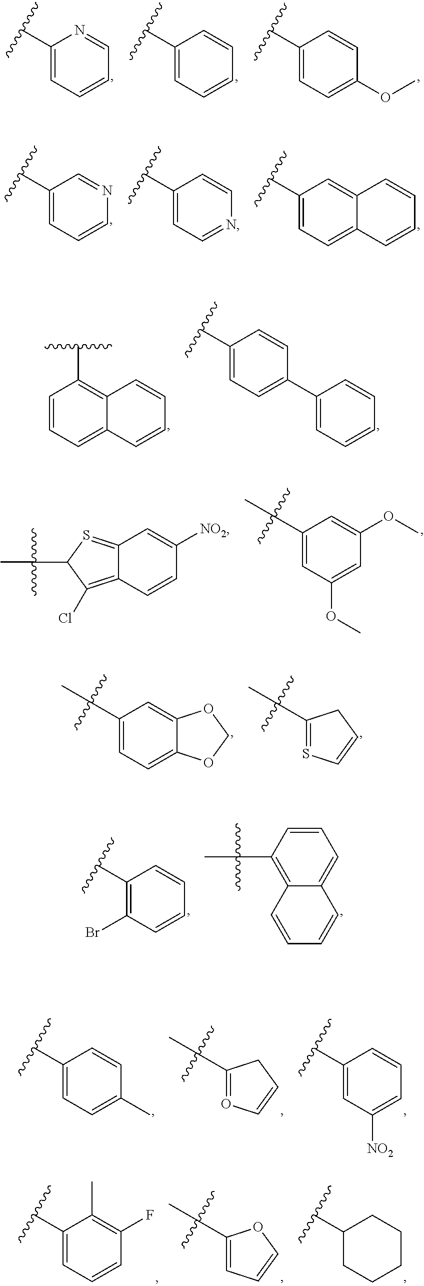

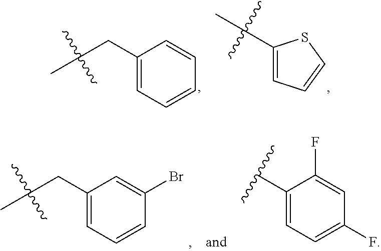

9. The composition of claim 6, wherein A is selected from the group consisting of: 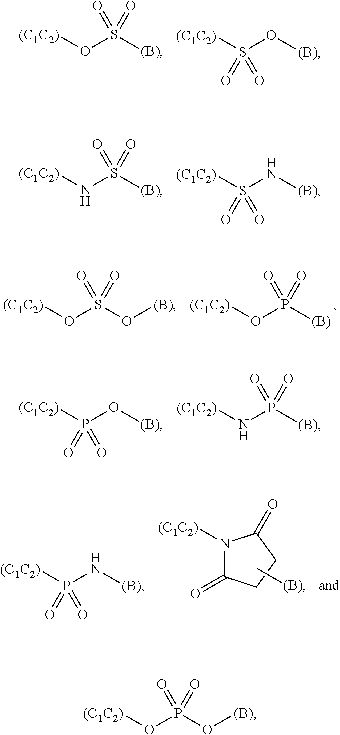
wherein X1 and X4 are present or absent and, if present, are independently selected from O and S, and
wherein X2, X3 and X5 are independently selected from O, C, S, SO₂, PO₂, and NH.

10. The composition of claim 6, wherein A is selected from the group consisting of: 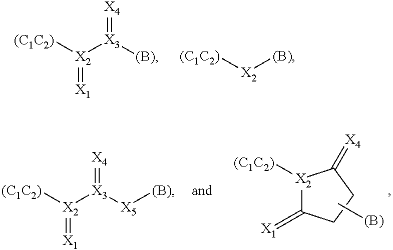
where X1 and X4 are present or absent and, if present, are independently selected from O and S, and where X2, X3 and X5 are independently selected from O, C, S, SO₂, PO₂, N, or NH.

11. The composition of claim 6, wherein C1C2 is selected from the group consisting of: 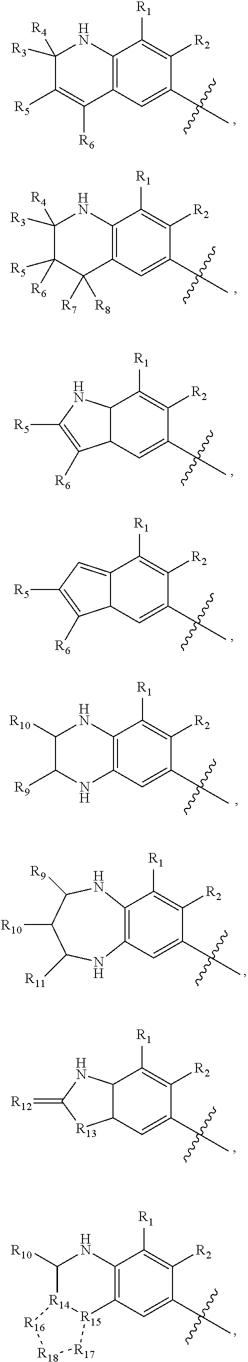 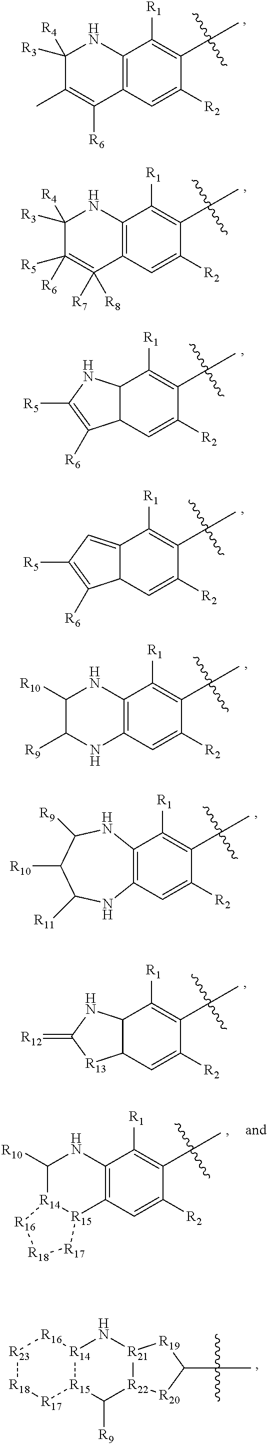
wherein R1 and R2 are independently selected from the group consisting of hydrogen, hydrogen-bond acceptors or donors,
wherein R3, R4, R5, R6, R7 and R8 can are independently selected from the group consisting of hydrogen, methyl, ethyl, iodo, bromo, chloro, and hydroxyl,
wherein R9, R10 and R11 are independently selected from the group consisting of halogen, CF₃, CI₃, CBr₃, CCl₃, COO^{-, COOH, ketone, hydrogen and methyl,}
wherein R12 and R13 are independently selected from the group consisting of O and S,
wherein R14 and 15 is either C═C or CH—CH,
where R16, R17, R18, and R23 are independently present or absent, and if present are independently selected from the group consisting of COO⁻, COOH, ketone, hydrogen, Cl, I, Br, F, CF₃, CI₃, CBr₃, CCl₃, C, CH, CH2, and methyl,
wherein R19 and R20 are independently selected from the group consisting of C, CH, CH2, N, and NH, and
wherein R21 and R22 is independently selected from the group consisting of C, CH and N.

12. The composition of claim 6, wherein B is selected from the group consisting of: 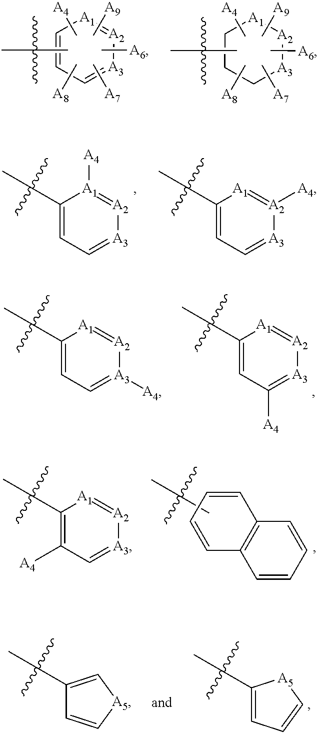
where A1, A2, and A3 are independently selected from C and N,
where A4, A6, A7, A8, and A9 are independently present or absent, and if present, are independently Cl, I, Br, F), CF₃, CI₃, CBr₃, CCl₃, 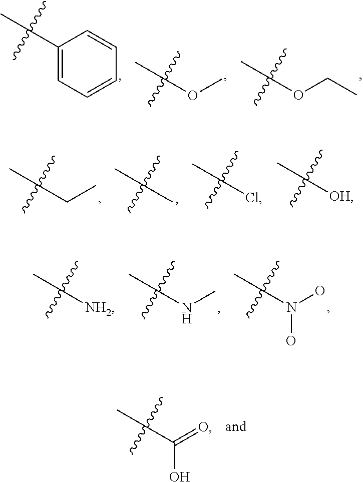
where A5 is selected from O, C, S, or NH.

13. The composition of claim 6, wherein C1 is non-aromatic.

14. The composition of claim 1, wherein the compound is selected from the group consisting of: 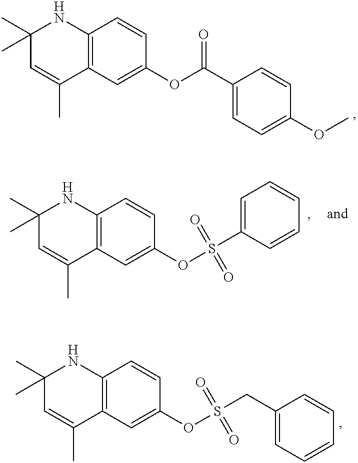
including salts, esters, and prodrugs thereof, and including both R and S enantiomeric forms and racemic mixtures thereof.

15. A method for inhibiting the activity of HIV protease, comprising binding one or more compounds of claim 1.

16. A method of treating a disorder associated with HIV protease, comprising administering to a subject suffering from an HIV protease associated disorder a therapeutic amount of one or more compounds of claim 1.

17. The method of claim 16, wherein said disorder associated with HIV protease is selected from the group consisting of: AIDS, AIDS Related Complex, and HIV Infection.

18. The method of claim 16, further comprising administering to said subject therapeutic amounts of one or more therapeutic agents known to treat disorders associated with HIV protease.

19. The method of claim 18, wherein said one or more therapeutic agents known to treat disorders associated with HIV protease is selected from the group consisting of a nucleotide reverse transcriptase inhibitor, a non-nucleoside reverse transcriptase inhibitor, a protease inhibitor, and an antiviral agent.

20. The method of claim 18, wherein said one or more therapeutic agents known to treat disorders associated with HIV protease is selected from the group consisting of zidovudine (AZT); didanosine (ddI); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)—FTC]; beta-L-FD4 (also called beta-L-D4C and named beta-L-2′,3′-dicleoxy-5-fluoro-cytidene); DAPD, the purine nucleoside, (−)-beta-D-2,6,-diamino-purine dioxolane; and lodenosine (FddA)), nevirapine (BI-RG-587); delaviradine (BHAP, U-90152); efavirenz (DMP-266) a benzoxazin-2-one; PNU-142721, a furopyridine-thio-pyrimide; 5-(3,5-dichlorophenyl)-thio-4-isopropyl-1-(4-pyridyl)methyl-I H-imidazol-2-ylmethyl carbonate; MKC442 (1-(ethoxymethyl)-5-(1-methylethyl)-6-(phenylmethyl)-(2,4(1H,3H)-pyrimidi-nedione); and (+)-calanolide A (NSC-675451) and B, coumarin derivatives), saquinavir (Ro 31-8959); ritonavir (ABT-538); nelfnavir (AG-1343); amprenavir (141W94); lasinavir (BMS-234475); DMP-450; ABT-378; and AG-1549), hydroxyurea, ribavirin, IL-2, IL-12, pentafuside, hydroxyurea (Droxia), IL-2, and pentafuside (DP-178, T-20).

21. A method of screening for compounds able to bind a region of HIV protease defined by the following residues: I84, V32, P81, T80, P79, G78, V56, I54, I47, G48, G49, and I50, comprising administering a candidate compound to a sample comprising HIV protease, and detecting binding of said compound within said HIV protease region.
